(19) United States
(12) Patent Application Publication
(10) Pub. No.: US 2026/0158177 A1
(43) Pub. Date: Jun. 11, 2026

(54) COMPOUNDS SPECIFIC TO GRANZYME B AND USES THEREOF

(71) Applicant: CYTOSITE BIOPHARMA INC., Sudbury, MA (US)

(72) Inventors: Hui XIONG, Indianapolis, IN (US); Carey HORCHLER, Indianapolis, IN (US); Mark A. CASTANARES, Indianapolis, IN (US); Brian LIEBERMAN, Indianapolis, IN (US); Juntian ZHANG, Indianapolis, IN (US); Francisco A. VALENZUELA, Indianapolis, IN (US)

(73) Assignee: CYTOSITE BIOPHARMA INC., Sudbury, MA (US)

(21) Appl. No.: 18/718,423

(22) PCT Filed: Dec. 7, 2022
(86) PCT No.: PCT/US2022/081098

§ 371 Date: Jun. 10, 2024

Related U.S. Application Data
(60) Provisional application No. 63/287,494, filed on Dec. 8, 2021.

(51) Int. Cl. A61K 51/04; A61K 31/555; A61K 31/56; A61K 38/00; A61K 51/08; A61P 35/00; C07K 7/06; C07K 16/00

(52) U.S. Cl. CPC A61K 51/0497; A61K 31/555; A61K 31/56; A61K 38/00; A61K 51/088; A61P 35/00; C07K 7/06; C07K 16/00

(57) ABSTRACT

Compounds capable of binding to granzyme B, for examples, compounds of Formula (I) and Formula (II), and pharmaceutical compositions comprising such. Also provided herein are uses of the compounds and pharmaceutical composition for imaging Granzyme B, in vivo or in vitro.

### CROSS-REFERENCE TO RELATED APPLICATIONS

This application claims the benefit of U.S. Provisional Application No. 63/287,494, filed Dec. 8, 2021, the content of which is incorporated by reference herein in its entirety.

### TECHNICAL FIELD

This disclosure relates to compounds useful for imaging techniques, and more particularly to compounds that are useful for imaging Granzyme B using medical imaging, including positron emission tomography.

### BACKGROUND

Granzyme B is a serine-protease most commonly found in the granules of natural killer cells and cytotoxic T cells. Granzyme B is released along with the pore-forming protein perforin at the immunological-synapse formed between T-cells and their targets. A portion of the released Granzyme B then enters cancer cells, primarily through perforin-pores, where it activates multiple substrates leading to activation of the caspase cascade. As a downstream effector of tumoral cytotoxic T cells, granzyme B has been used as an early biomarker for tumors responding to immunotherapy.

There is a need to develop new compounds that act as effective Granzyme B imaging agents, and therapies for treating immunoregulatory abnormality such as cancer.

### SUMMARY

The present disclosure is based, at least in part, on the development of granzyme B-binding compounds, which exhibit superior features, such as good in vivo imaging activity, no gut intake, and great renal clearance with no production of metabolites as observed in an animal model. Such granzyme B (GZB)-binding compounds can be used in GZB imaging (e.g., in vivo), the results of which can be relied for therapeutic and diagnostic purposes, for example, for identifying suitable patients for treatment and/or for monitoring treatment efficacy.

Accordingly, in one aspect, the present disclosure features a compound, or a pharmaceutically acceptable salt thereof, of formula (I):

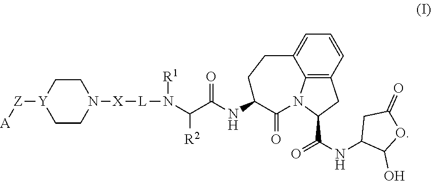

In formula (I),
A is a chelating moiety (e.g., those disclosed herein);
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—;
Y is CH or N;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—;
L is a peptide linker having 1-6 amino acid residues, inclusive;
R¹ is H or C₁₋₆ alkyl, optionally wherein R¹ is H or methyl; and
R² is C₁₋₆ alkyl or C₃₋₆ cycloalkyl.

In some examples, X can be —CH₂C(O)—. In other examples, X can be —NHC(S)—.

In some examples, Z may be —CH₂— (e.g., when the ring connecting to it is a piperidine ring). Alternatively, Z may be —CH₂C(O)— (e.g., when the ring connecting to it is a piperazine ring).

In some embodiments, R¹ is H and R² is C₄ alkyl, for example, as in compounds of formula (Ia):

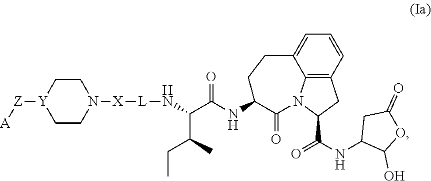

in which variables A, X, Y, Z, and L are as defined herein.

In further embodiments, X is —CH₂C(O)—, for example, as in compounds of formula (Ib):

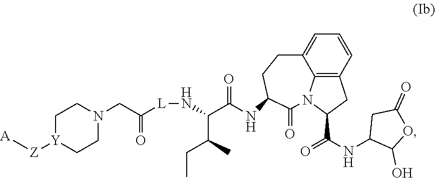

in which variable A, Y, Z, and L are as defined herein.

In some of the above embodiments, Y is —CH—, and Z is —CH₂—, for example, as in formula (Ib-A):

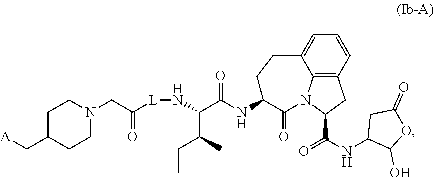

in which variables A and L each are as defined herein.

Exemplary Compounds of formula (Ib-A) include Compounds 1-7 as described herein.

In others of the above embodiments, Y is —N—, and Z is —CH₂C(O)—, for example, as in formula (Ib-B):

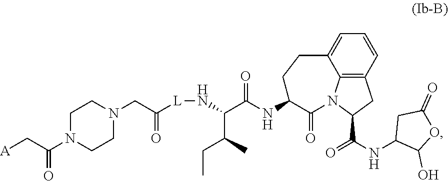

in which variable A and L each are as defined herein.

Exemplary Compounds of formula (Ib-B) include Compounds 8-17 as described herein.

In another aspect, the present disclosure features a compound, or a pharmaceutically acceptable salt thereof, of formula (II):

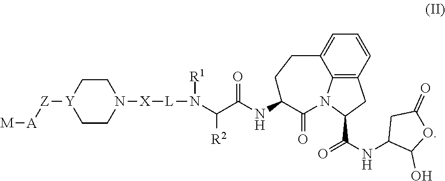

The variables of formula (II) may be described as:
M is a metal or a metal linked to a radioisotope;
A is a chelating moiety chelating the metal;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—;
Y is CH or N;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—;
L is a peptide linker having 1-6 amino acid residues, inclusive;
R¹ is H or C₁₋₆ alkyl, optionally wherein R¹ is H or methyl; and
R² is C₁₋₆ alkyl or C₃₋₆ cycloalkyl.

In some examples, X can be —CH₂C(O)—. In other examples, X can be —NHC(S)—.

In some examples, Z may be —CH₂— (e.g., when the ring connecting to it is a piperidine ring). Alternatively, Z may be —CH₂C(O)— (e.g., when the ring connecting to it is a piperazine ring).

In some embodiments, R¹ is H and R² is C₄ alkyl, for example, as in compounds of formula (IIa):

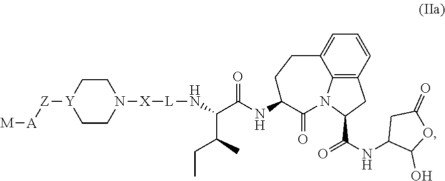

in which variables M, A, X, Y, Z, and L are as defined herein.

In further embodiments, X is —CH₂C(O)—, for example, as in compounds of formula (IIb):

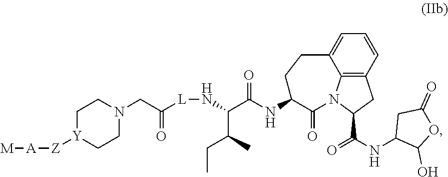

in which variables M, A, Y, Z, and L are as defined herein.

In some embodiments, Y is —CH—, and Z is —CH₂—, for example, as in formula (IIb-A):

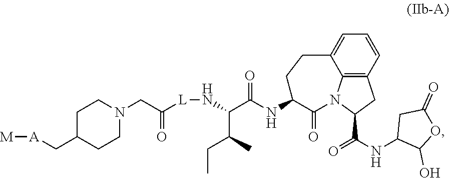

in which variables M, A, and L each are as defined herein.

In other embodiments, Y is —N—, and Z is —CH₂C(O)—, for example, as in formula (IIb-B):

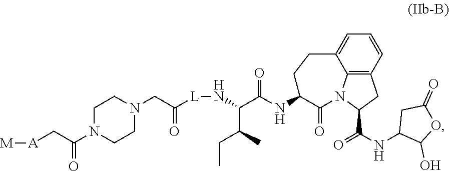

in which variables M, A, and L each are as defined herein.

In any of the GZB-binding compounds disclosed herein, L may have 1-6 amino acid residues, for example, 1-5 amino acid residues. The amino acid residues may be standard proteinogenic amino acids (i.e., the 20 naturally-occurring amino acid residues found in naturally-occurring proteins), or unnatural amino acids, which may be derivatives of a natural-occurring protein or an isomer of a naturally-occurring amino acid residue. Structures of exemplary non-naturally occurring amino acid residues that may be included in the L linker are provided in Table 1 below. Exemplary amino acid sequences include Glu-Gly-Gly, Glu-βAla-βAla, γGlu, _{D}γGlu, γGlu-βAla, _{D}Glu-βAla-βAla, _{D}Glu-AEA, _{D}Glu-AEEA-AEEA, _{D}Glu-_{D}Glu-AEA, _{D}Glu-_{D}Glu-βAla-βAla, βAla-_{D}Glu-βAla, Diacid-βAla-βAla, N-acid-βAla-βAla, and βAla-N-acid-βAla. See Table 2 for structures of these exemplary L linkers.

In any of the compounds of formula (II) disclosed herein, M may be a metal known in the art to be useful as described herein, e.g., for imaging purposes. In some examples, the metal (either alone or in conjugation with the radioisotope) may be toxic. In other examples, the metal (either alone or in conjunction with the radioisotope) may be non-toxic. Exemplary metals include Ga (e.g., ⁶⁸Ga) and Al (which may be conjugated to an isotope such as ¹⁸F).

In any of the compounds of formula (I) or (II) described herein, A may be a chelating moiety known in the art to be useful as described herein, e.g., to bind a metal. In some examples, the chelating moiety is 1,4,7-triazacyclononane-N,N′,N″-triacetic acid (NOTA) or 1,4,7-triazacyclononane-4,7-diyl diacetic acid (NODA).

In another aspect, the present disclosure features a pharmaceutical composition comprising any of the GZB-binding compounds disclosed herein, a stereoisomer thereof, or a pharmaceutically acceptable salt thereof, and a pharmaceutically acceptable carrier.

In another aspect, the present disclosure features a method of imaging granzyme B in a tissue. The method comprises:
(i) contacting any of the formula (II) compounds disclosed herein, or the pharmaceutically acceptable salt thereof, with a tissue suspected of comprising granzyme B, and
(ii) imaging the tissue based on radioisotope signals released from the compound, or the pharmaceutically acceptable salt thereof.

In some embodiments, the imaging method disclosed herein is performed in vitro. For example, the tissue for imaging by the method is in a biological sample, which may be obtained from a subject (e.g., a human patient) as disclosed herein.

In other embodiments, the imaging method disclosed herein can be performed in vivo, wherein an effective amount of the compound or the pharmaceutically acceptable salt thereof may be administered to a subject (e.g., a human patient) in need thereof.

In some embodiments, the subject is on a treatment (e.g., anti-inflammatory agents, steroids, immunotherapy agents, chemotherapeutic agents, and therapeutic antibodies) for an immunoregulatory abnormality (e.g., an autoimmune disorder, an inflammatory disorder, a skin disorder, a cancer, and a cardiovascular disorder)

In some embodiments the immune response in the subject is monitored based on the imaging of granzyme B.

Also provided herein are any of the compounds of formula (II), a pharmaceutically acceptable salt thereof, or a pharmaceutical composition comprising such for use in imaging granzyme B for diagnostic purposes or for monitoring treatment efficacy.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 depicts semiprep HPLC Radio-trace for preparing Compound ¹⁸F-1-Al.

FIG. 2 depicts analytical HPLC Radio-trace for Compound ¹⁸F-1-Al.

FIG. 3 depicts semiprep HPLC Radio-trace for preparing Compound ¹⁸F-12-Al.

FIG. 4 depicts analytical HPLC Radio-trace for Compound ¹⁸F-12-Al.

FIGS. 5A-5B include photos showing in vivo imaging activity of exemplary GZB-binding compounds disclosed herein. FIG. 5A: Exemplary Compound ¹⁸F-12-Al relative to reference Compound ¹⁸F-18-Al. FIG. 5B: Exemplary Compounds ¹⁸F-19-Al, ¹⁸F-20-Al, ¹⁸F-8-Al, and ¹⁸F-2-Al relative to reference Compound ¹⁸F-18-Al. FIG. 5C: Exemplary Compounds ¹⁸F-12-Al, ¹⁸F-10-Al, and ¹⁸F-13-Al relative to reference Compound ¹⁸F-18-Al. FIG. 5D: Exemplary Compounds ¹⁸F-1-Al, ¹⁸F-4-Al, and ¹⁸F-3-Al relative to reference Compound ¹⁸F-18-Al.

FIGS. 6A and 6B include diagrams showing quantified in vivo imaging activity of exemplary GZB-binding compounds having the piperazine rind or the piperidine ring. FIG. 6A: percentage of injected dose per gram (% ID/g). FIG. 6B: Target to background ratios (TBR). Columns from left to right: reference Compound ¹⁸F-18-Al, Compound ¹⁸F-12-Al, Compound ¹⁸F-10-Al, Compound ¹⁸F-13-Al, Compound ¹⁸F-1-Al, Compound ¹⁸F-4-Al, and Compound ¹⁸F-3-Al.

### DETAILED DESCRIPTION

Cancer immunotherapies have represented a significant advance in cancer therapy over recent years. Antibodies directed against immune checkpoints such as programmed cell death protein 1 (PD-1) and cytotoxic t lymphocyte-associated protein 4 (CTLA-4) have been approved with positive outcomes for some patients. Research into the field of immune-oncology continues, with strategies including CAR-T cells, vaccines, small molecules, and antibodies under development. Despite the promise of these therapies, they are not a panacea. These immunotherapies can be associated with significant adverse events, which are costly, and the response rates are typically 20-50%, meaning the majority of patients do not respond to therapy. Furthermore, determining an individual patient's response to therapy can be challenging using conventional methods, as response is frequently associated with an immune-cell infiltrate that can make responding tumors appear to grow on anatomic imaging (e.g., CT, MRI) and demonstrate increased avidity with FDG-PET imaging due to the influx of metabolically active immune cells. Given the constraints of current imaging technologies, clinical studies for cancer immunotherapies typically employ overall survival as their study endpoint as opposed to progression-free survival.

Granzyme B, a downstream marker of cytotoxic T-cell activity, could serve as a novel biomarker to assess cancer immunotherapy efficacy. Granzyme B expression within a tumor can be assessed not only for CTL presence or absence, but also as an effector protein released by active T-cells that also integrates a measure of CTL activity, thus accounting for issues of T-cell exhaustion that make assessment of CTL presence difficult to accomplish.

The present disclosure provides certain specific compounds capable of binding to Granzyme B (GZB), e.g., Formula (I) compounds and Formula (II) compounds, which show high binding affinity to Granzyme B. Such compounds have shown superior features, including good in vivo imaging activity, little or no gut intake, and complete renal clearance with no production of metabolites. Accordingly, the GZB-binding compounds disclosed herein can serve as Granzyme B imaging agents with high sensitivity and specificity (e.g., low background signal). The GZB-binding compounds disclosed herein can be used to identify patients who are responsive to an immunotherapeutic agent or monitoring treatment efficacy of an immunotherapeutic agent based on the level and/or location of Granzyme B as determined by the imaging assay disclosed herein.

### Definitions

It is to be understood that the terminology employed herein is for the purpose of describing particular embodiments and is not intended to be limiting. Further, although any methods, devices, and materials similar or equivalent to those described herein can be used in the practice or testing of the invention, the preferred methods, devices, and materials are now described. In addition to the foregoing, as used in the specification and appended claims, unless specified to the contrary, the following terms have the meaning indicated:
“Amino” refers to the —NH₂ radical.
“Cyano” refers to the —CN radical.
“Hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.
“Trifluoromethyl” refers to the —CF₃ radical.
“Alkyl” refers to a linear, saturated, acyclic, monovalent hydrocarbon radical or branched, saturated, acyclic, monovalent hydrocarbon radical, having from one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylpenty-1,2-methylpentyl and the like. An alkyl moiety may be unsubstituted. Alternatively, an alkyl moiety may be optionally substituted. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR³, —OC(O)—R³, —N(R³)₂, —C(O)R⁴, —C(O)OR³, —C(O)N(R³)₂, —N(R³)C(O)OR⁵, —N(R³)C(O)R⁵, —N(R³)S(O)ₜR⁵ (where t is 1 or 2), —S(O)ₜOR⁵ (where t is 1 or 2), —S(O)ₚR⁵ (where p is 0, 1, or 2) and —S(O)ₜN(R³)₂ (where t is 1 or 2), where each R³ is independently hydrogen, alkyl, haloalkyl, cycloalkyl, cycloalkylalkyl, aryl, aralkyl, heterocyclyl, or heteroaryl; each R⁴ is independently hydrogen, cycloalkyl, aryl, heterocyclyl, or heteroaryl; and each R⁵ is independently alkyl, haloalkyl, cycloalkyl, aryl, heterocyclyl, or heteroaryl.
“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. A cycloalkyl moiety may be unsubstituted. Alternatively, a cycloalkyl moiety may be optionally substituted. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R⁴—OR³, —R⁴—OC(O)—R³, —R⁴—N(R³)₂, —R⁴—C(O)R³, R⁴—C(O)OR³, —R⁴—C(O)N(R³)₂, —R⁴—N(R³)C(O)OR⁵, —R⁴—N(R³)C(O)R⁵, —R⁴—N(R³)S(O)ₜR⁵ (where t is 1 or 2), —R⁴—S(O)ₜOR⁵ (where t is 1 or 2), —R⁴—S(O)ₚR⁵ (where p is 0, 1, or 2) and —R⁴—S(O)ₜN(R³)₂ (where t is 1 or 2) where each R³ is independently hydrogen, alkyl, haloalkyl, cycloalkyl, aryl, heterocyclyl, or heteroaryl; each R⁴ is independently a direct bond or a linear or branched alkylene or alkenylene chain; and each R⁵ is independently alkyl, haloalkyl, cycloalkyl, cycloalkylalkyl, aryl, aralkyl, heterocyclyl, or heteroaryl.

“Chelating moieties” are those molecules or ions, which are able to act as a polydentate ligand to a metal ion. For example, molecules with multiple atoms with available lone pairs (including but not limited to nitrogen and oxygen) may act as chelating moieties. Chelating moieties may be linear (e.g., EDTA), or cyclic (including macrocycles e.g., DOTA, porphyrin) and may involve macrocyas commonly known in the art. Chelating moieties may have 2, 3, 4, 5, or 6 functional groups (e.g., amines, amides, hydroxyls, carboxylic acids etc.) with available lone pairs to coordinate with a metal.

In some embodiments, preparation of compounds can involve the addition of acids or bases to affect, for example, catalysis of a desired reaction or formation of salt forms such as acid addition salts.

Exemplary acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Exemplary bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

As used herein, the phrase “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

### I. Granzyme B-Targeting Compounds

In some aspects, provided herein are Granzyme B-targeting compounds disclosed herein, e.g., Formula (I) or Formula (II) compounds. The compounds disclosed herein encompass the compounds per se, their pharmaceutically acceptable salt thereof, and stereoisomers thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### A. Compounds of Formula (I)

In some embodiments, the disclosure provides compounds of Formula (I), shown below, which are capable of binding to Granzyme B with high binding affinity and/or specificity:

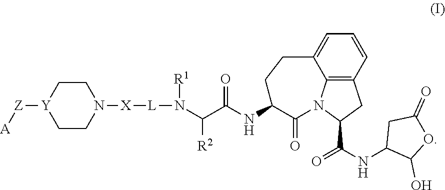

In Formula (I), A is a chelating moiety. Exemplary chelating moieties for use in the Granzyme B-targeting compounds disclosed herein include, but are not limited to, 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), 1,4,7-triazacyclononane-4,7-diyl diacetic acid (NODA) and Desferrioxamine B (DFO). In some embodiments, the chelating agent is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-4,7-diyl diacetic acid (NODA) and 1,4,7-triazacyclononane-1-glutaric acid-4,7-diacetic acid (NODAGA). In some embodiments, the chelating agent is 1,4,7-triazacyclononanetriacetic acid (NOTA). In other embodiments, the chelating agent is 1,4,7,10-tetraazacyclododecane-1,4,7,10-tetraacetic acid (DOTA). In some embodiments, the chelating agent is 1,4,7-triazacyclononane-4,7-diyl diacetic acid (NODA).

X can be —CH₂C(NH)—, —CH₂C(O)—, —CH₂C(S)—, —NHC(NH)—, —NHC(O)—, —NHC(S)—, —OC(NH)—, —OC(O)—, or —OC(S)—. In one example, X is —CH₂C(O)—. In another example, X is —NHC(S)—.

In some embodiments, Y is CH, forming a piperidine ring. In other embodiments, Y is N forming a piperazine ring.

Z can be —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—. In some embodiments, Z is —CH₂—. In other embodiments, Z is —CH₂C(O)—.

L can be a peptide linker having 1-6 amino acid residues, inclusive. In some examples, L includes 1-5 amino acid residues, inclusive. In other examples, L includes 2-4 amino acid residues, inclusive. In one example, L includes 1 amino acid residue. In another example, L includes 2 amino acid. In yet another example, L includes 3 amino acid residues. Alternatively, L includes 4 amino acid residues. In still another example, L includes 5 amino acid residues. Alternatively, L includes 6 amino acid residues.

Compatible amino acid residues in the peptide linker L may include natural and non-natural amino acid residues (including β-amino acid residues and D-amino acids) and is not limited to protienogenic amino acid residues. As used herein, proteinogenic amino acid residues refer to the 20 amino acid residues existing in nature as building blocks for synthesizing proteins. Amino acid residues may form a chain through standard peptide bonds, or by forming amide bonds with compatible side chains (e.g., glutamic acid (e.g., D-Glu), aspartic acid). Table 1 provides some exemplary non-proteinogenic (non-naturally occurring) amino acids that can be used in any of the peptide linker L disclosed herein, including their chemical structures.

TABLE 1
Exemplary Non-Proteinogenic Amino Acids

| Chemical Structure | Non-Naturally Occurring Amino Acids |
|---|---|
| 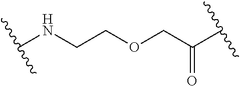 [SMILES: *NCCOCC(*)=O] | AEA |
| 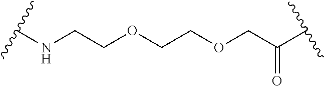 [SMILES: *NCCOCCOCC(*)=O] | AEEA |
| 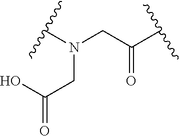 [SMILES: *C(=O)CN(*)CC(=O)O] | N-acid |
| 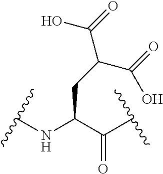 [SMILES: *N[C@@H](CC(C(=O)O)C(=O)O)C(*)=O] | Diacid |
| 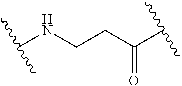 [SMILES: *NCCC(*)=O] | βAla |
| 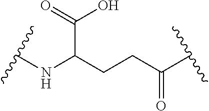 [SMILES: *NC(CCC(*)=O)C(=O)O] | γGlu |

Exemplary peptide linkers include, but are not limited to, Glu-Gly-Gly, Glu-βAla-βAla, γGlu, _{D}γGlu, γGlu-βAla, _{D}Glu-βAla-βAla, _{D}Glu-AEA, _{D}Glu-AEEA-AEEA, _{D}Glu-_{D}Glu-AEA, _{D}Glu-_{D}Glu-βAla-βAla, βAla-_{D}Glu-βAla, Diacid-βAla-βAla, N-acid-βAla-βAla, and βAla-N-acid-βAla.

TABLE 2
Exemplary Peptide Linkers

| | |
|---|---|
| 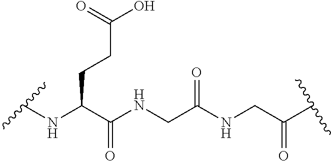 [SMILES: *N[C@@H](CCC(=O)O)C(=O)NCC(=O)NCC(*)=O] | Glu-Gly-Gly |
| 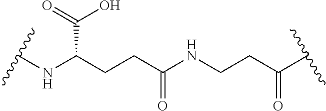 [SMILES: *N[C@@H](CCC(=O)NCCC(*)=O)C(=O)O] | γGlu-βAla |
| 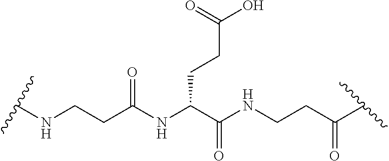 [SMILES: *NCCC(=O)N[C@H](CCC(=O)O)C(=O)NCCC(*)=O] | βAla-_{D}Glu-βAla |
| 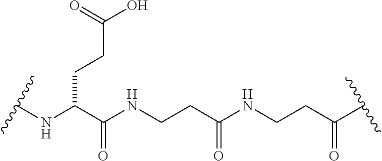 [SMILES: *N[C@H](CCC(=O)O)C(=O)NCCC(=O)NCCC(*)=O] | Glu-βAla-βAla |
| 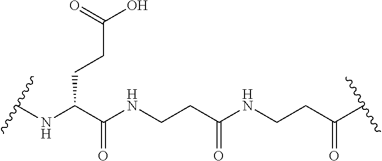 [SMILES: *N[C@H](CCC(=O)O)C(=O)NCCC(=O)NCCC(*)=O] | _{D}Glu-βAla-βAla |
| 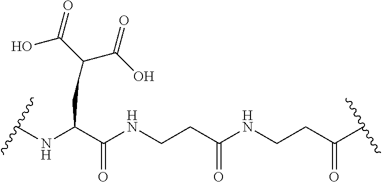 [SMILES: *N[C@@H](CC(C(=O)O)C(=O)O)C(=O)NCCC(=O)NCCC(*)=O] | Diacid-βAla-βAla |
| 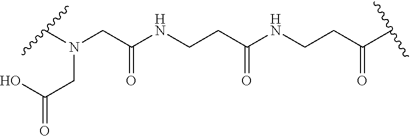 [SMILES: *C(=O)CCNC(=O)CCNC(=O)CN(*)CC(=O)O] | N-acid-βAla-βAla |
| 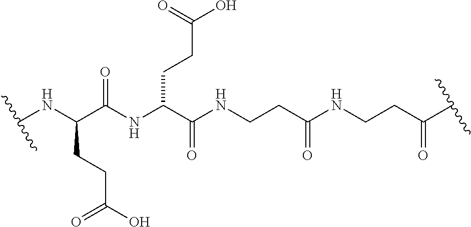 [SMILES: *N[C@H](CCC(=O)O)C(=O)N[C@H](CCC(=O)O)C(=O)NCCC(=O)NCCC(*)=O] | _{D}Glu-_{D}Glu-βAla-βAla |
| 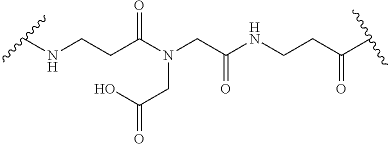 [SMILES: *NCCC(=O)N(CC(=O)O)CC(=O)NCCC(*)=O] | βAla-N-acid-βAla |
| 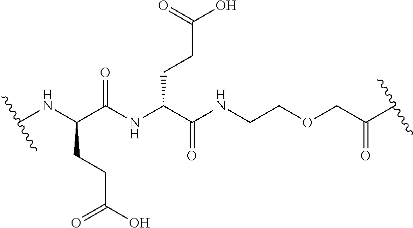 [SMILES: *N[C@H](CCC(=O)O)C(=O)N[C@H](CCC(=O)O)C(=O)NCCOCC(*)=O] | _{D}Glu-_{D}Glu-AEA |
| 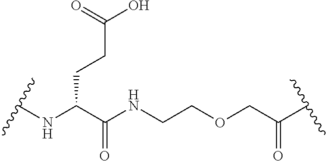 [SMILES: *N[C@H](CCC(=O)O)C(=O)NCCOCC(*)=O] | _{D}Glu-AEA |
| 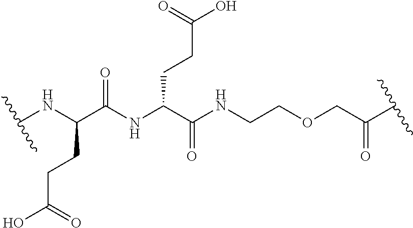 [SMILES: *N[C@H](CCC(=O)O)C(=O)N[C@H](CCC(=O)O)C(=O)NCCOCC(*)=O] | _{D}Glu-_{D}Glu-AEA |
| 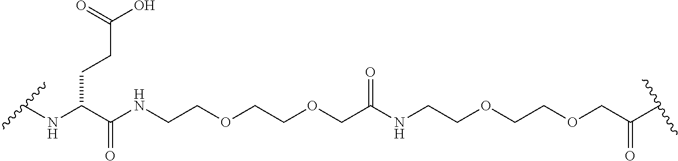 [SMILES: *N[C@H](CCC(=O)O)C(=O)NCCOCCOCC(=O)NCCOCCOCC(*)=O] | _{D}Glu-AEEA-AEEA |
| 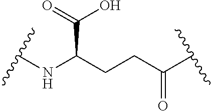 [SMILES: *N[C@H](CCC(*)=O)C(=O)O] | γGlu |
| 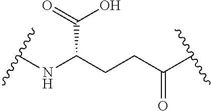 [SMILES: *N[C@@H](CCC(*)=O)C(=O)O] | _{D}γGlu |

In some embodiments, R¹ is H. In other embodiments, R¹ is C₁₋₆ alkyl. For example, R¹ can be methyl.

In some embodiments, R² can be C₁₋₆ alkyl. Alternatively, R² can be C₃₋₆ alkyl (e.g., branched or unbranched, substituted or unsubstituted) or C₃₋₆ cycloalkyl (e.g., cyclopropyl, cyclobutyl, cyclopentyl, cyclohexyl).

The hemiacetal unit in a compound of Formula (I) can be in an open chain aldehyde form. As a result, the compound of formula (I), can have the following structure:

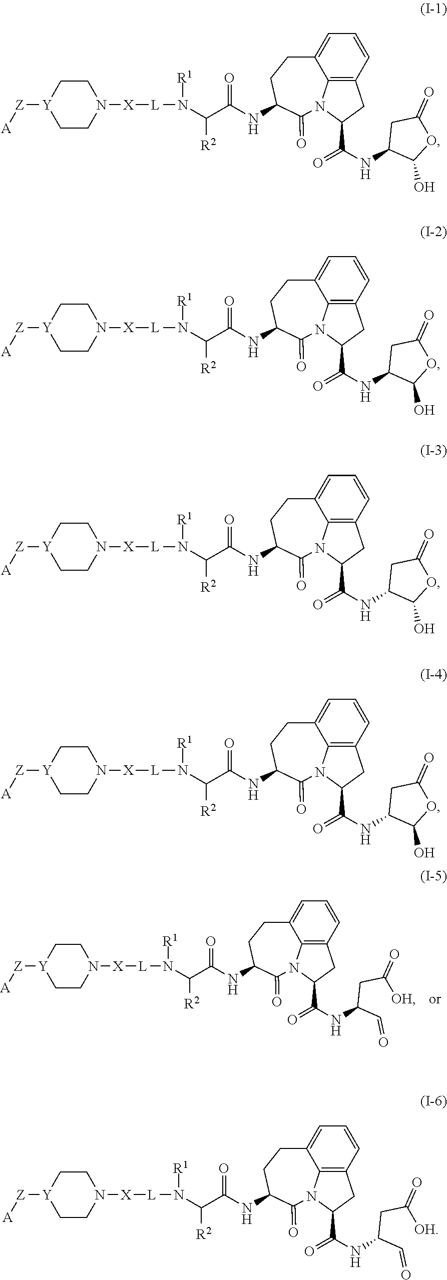

In any of Formulae (I-1) to (I-6), each of A, X, Y, Z, L, R¹ and R² is as described herein.

In some embodiments, R¹ is H, and R² is C₄ alkyl as in compounds of Formula (Ia):

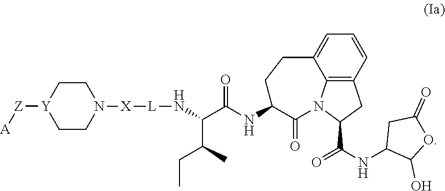

In some specific examples, X is —CH₂C(O)—, as in compounds of Formula (Ib):

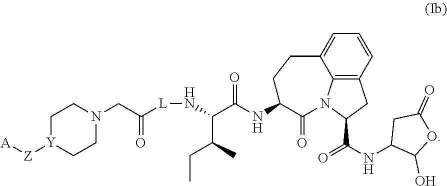

In some examples of Formula (Ib), Y is —CH—, and Z is CH₂ as in compounds of Formula (Ib-A):

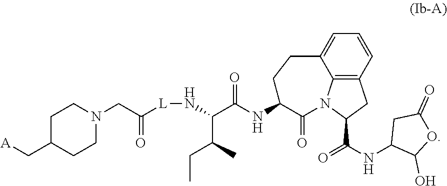

Exemplary compounds of Formula (I) include those listed in Table 3A.

TABLE 3A
Exemplary Compounds of Formula (Ib-A) (Piperidine Compounds)

| Compounds | ID |
|---|---|
| 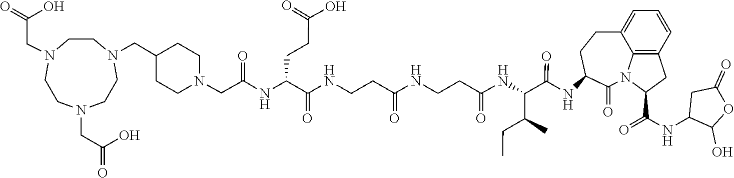 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 1 |
| 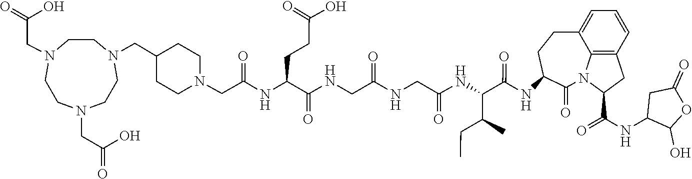 [SMILES: CC[C@H](C)[C@H](NC(=O)CNC(=O)CNC(=O)[C@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 2 |
| 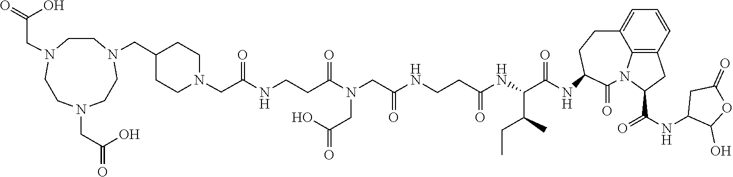 [SMILES: CC[C@H](C)[C@H](NC(=O)CCNC(=O)CN(CC(=O)O)C(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 3 |
| 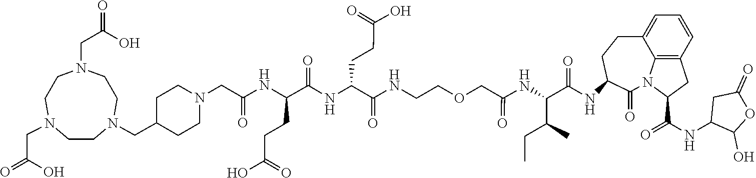 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 4 |
| 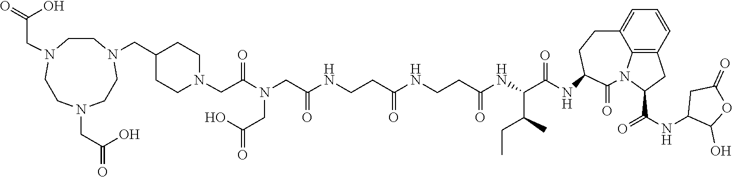 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 5 |
| 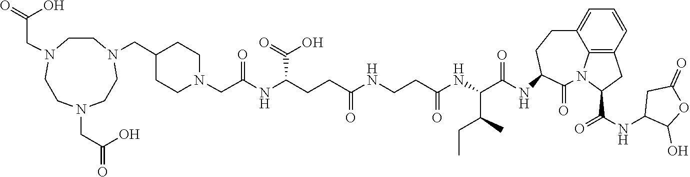 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3] | 6 |
| 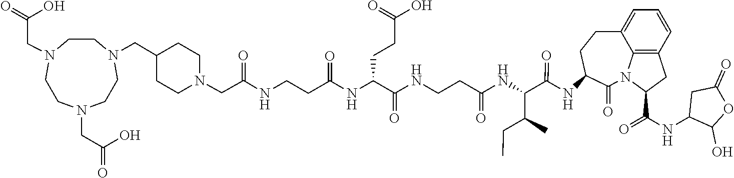 [SMILES: CC[C@H](C)[C@H](NC(=O)CCNC(=O)[C@@H](CCC(=O)O)NC(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 7 |

In some examples, the compound of Formula (I) is Compound 1, 2, 3, 4, 5, 6, or 7. In one specific example, the compound of Formula (I) is Compound 1. In another specific example, the compound of Formula (I) is Compound 4. As described herein, the above examples benefit from the piperidine ring, which exhibits improved properties over other related moieties.

The present application also includes stereoisomers of the compounds, such as stereoisomers of Compound 1 described herein. The stereoisomers result from the two chiral centers (closed ring) or one chiral center (open chain) of the hemiacetal unit as indicated by the wiggle lines:

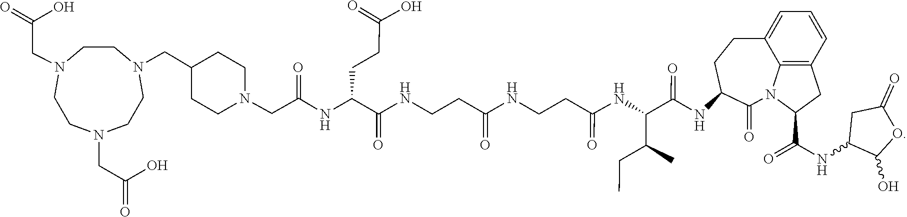 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Examples of the stereoisomers of Compound 1 is listed in Table 3B below.

TABLE 3B
Stereoisomers of Compounds 1

| Compounds | ID |
|---|---|
| 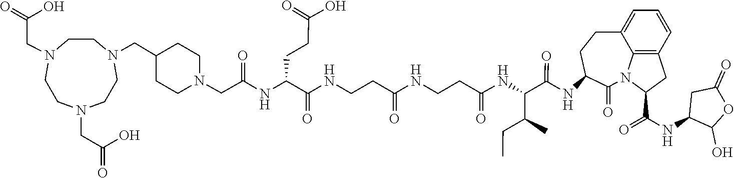 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)OC1O)C3] | 1-(S) |
| 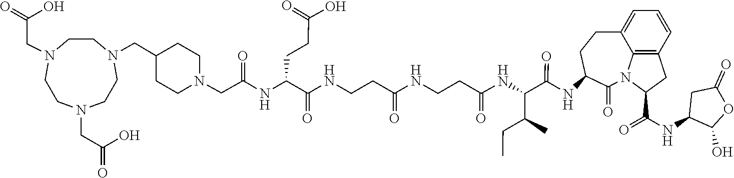 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@@H]1O)C3] | 1-(S)(S) |
| 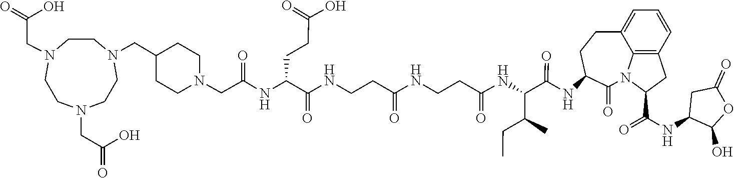 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1O)C3] | 1-(S)(R) |
| 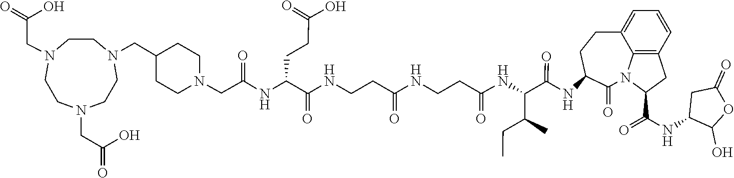 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)OC1O)C3] | 1-(R) |
| 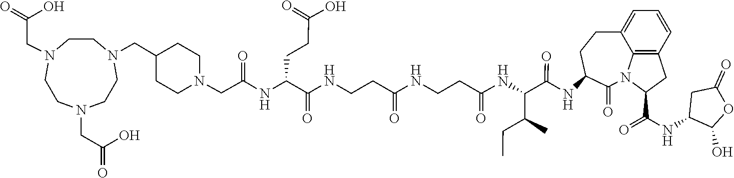 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@@H]1O)C3] | 1-(R)(S) |
| 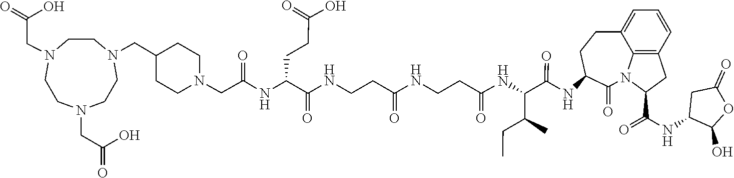 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@H]1O)C3] | 1-(R)(R) |

In one specific example, the compound of Formula (I) is Compound 1, stereoisomer 1-(S).

In other examples of Formula (Ib), Y is —N—, and Z is —CH₂C(O)— as in compounds of Formula (Ib-B):

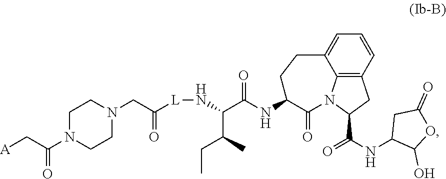

Exemplary compounds of Formula (I) include those listed in Table 4.

TABLE 4
Exemplary Compounds of Formula (Ib-B) (Piperazine Compounds)

| Compounds | ID |
|---|---|
| 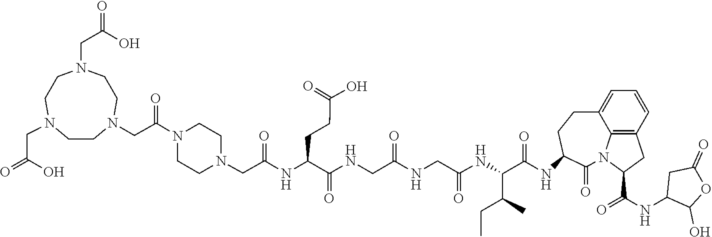 [SMILES: CC[C@H](C)[C@H](NC(=O)CNC(=O)CNC(=O)[C@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 8 |
| 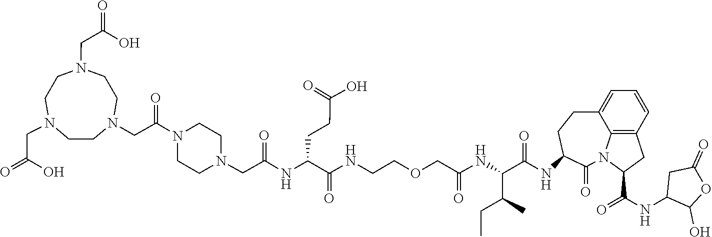 [SMILES: CC[C@H](C)[C@H](NC(=O)COCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 9 |
| 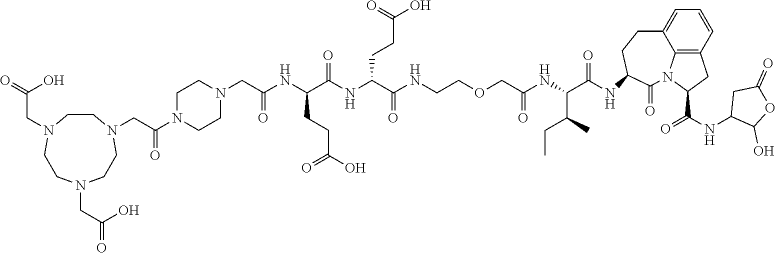 [SMILES: CC[C@H](C)[C@H](NC(=O)COCCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 10 |
| 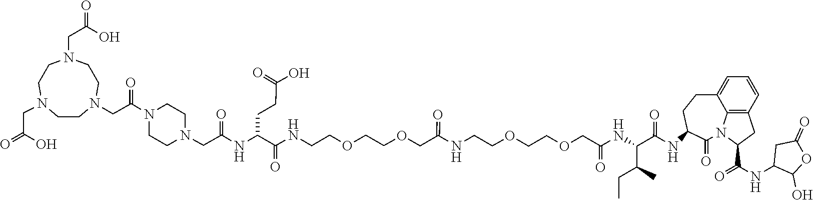 [SMILES: CC[C@H](C)[C@H](NC(=O)COCCOCCNC(=O)COCCOCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 11 |
| 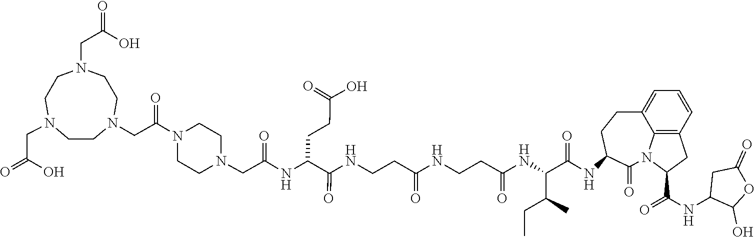 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 12 |
| 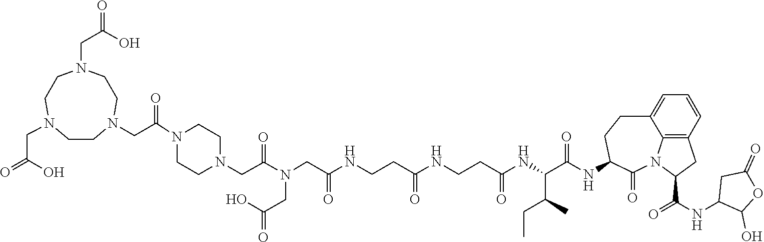 [SMILES: CC[C@H](C)[C@H](NC(=O)CCNC(=O)CCNC(=O)CN(CC(=O)O)C(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 13 |
| 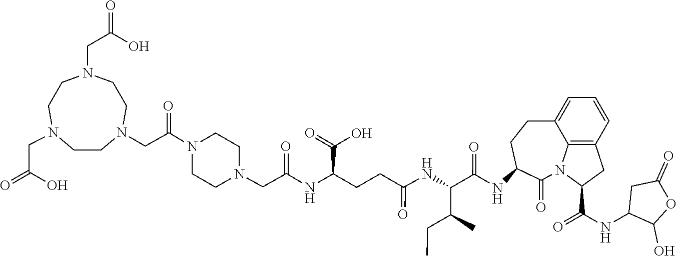 [SMILES: CC[C@H](C)[C@H](NC(=O)CC[C@@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3] | 14 |
| 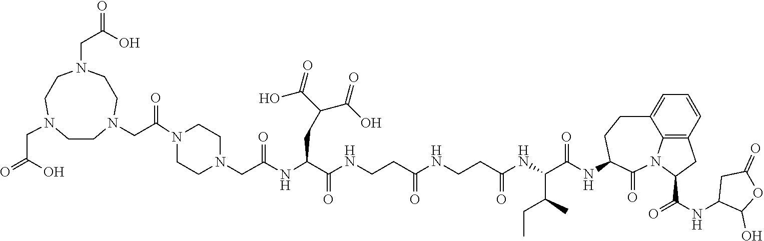 [SMILES: CC[C@H](C)[C@H](NC(=O)CCNC(=O)CCNC(=O)[C@H](CC(C(=O)O)C(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 15 |
| 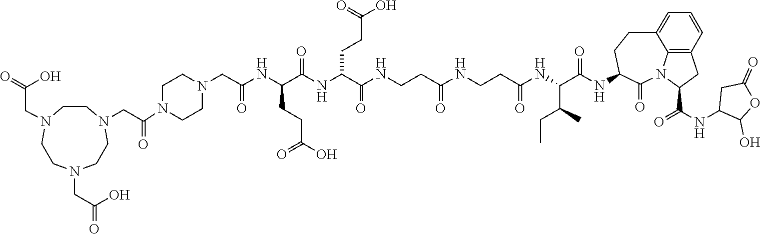 [SMILES: CC[C@H](C)[C@H](NC(=O)CCNC(=O)CCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3] | 16 |
| 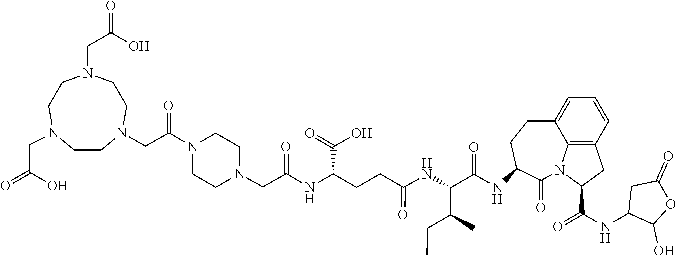 [SMILES: CC[C@H](C)[C@H](NC(=O)CC[C@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3] | 17 |

In some examples, the compound of Formula (I) is Compound 8, 9, 10, 11, 12, 13, 14, 15, 16, or 17. As described herein, the above examples benefit from the piperazine ring, which exhibits improved properties over many other related moieties.

### B. Compounds of Formula (II)

In some embodiments, the disclosure provides for compounds of Formula (II), shown below. Compared to the Formula (I) compounds disclosed herein, the Formula (II) compounds further contain a metal, which may be conjugated to a radioisotope, via the chelating moiety A.

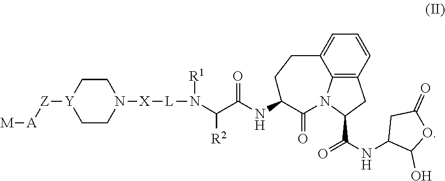

In Formula (II), M is a metal, or a metal linked to a radioisotope. Suitable metals for use in the present disclosure include those which are useful in imaging Granzyme B, for instance metals that are suitable radioimaging agents, as well as metals that can bind non-metal radioisotopes which are suitable radioimaging agents. An exemplary metal radioisotope is ⁶⁸Ga. An exemplary non-metallic radioisotope is ¹⁸F, which may be conjugated with Al for loading into the Granzyme B binding compounds disclosed herein

Each of A, X, Y. Z. L, R¹, and R² is as defined herein. See, e.g., the section titled Compounds of Formula (I) above.

In some embodiments, R¹ is H, and R² is C₄ alkyl as in compounds of Formula (IIa):

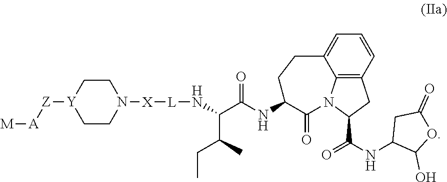

In some specific examples, X is —CH₂C(O)—, as in compounds of Formula (IIb):

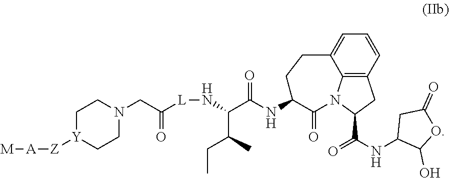

In some examples of Formula (IIb), Y is —CH—, and Z is CH₂ as in compounds of Formula (IIb-A):

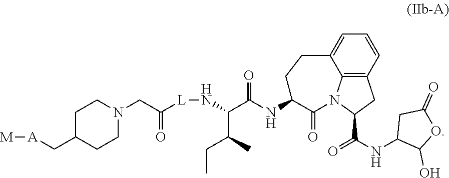

Exemplary compounds with radioisotopes include those in Table 5.

TABLE 5
Exemplary Compounds of Formula (IIb-A)

| Compounds | ID |
|---|---|
| 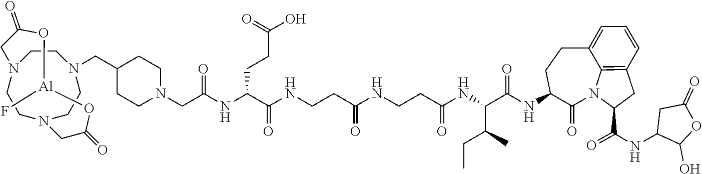 | 1-Al |
| 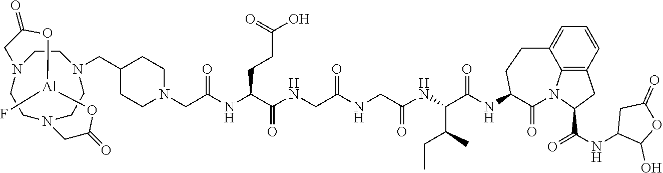 | 2-Al |
| 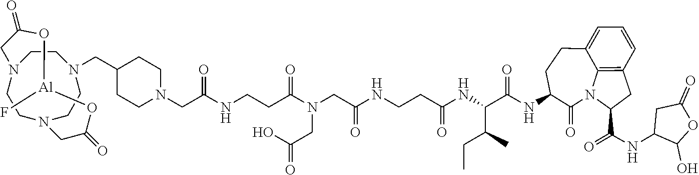 | 3-Al |
| 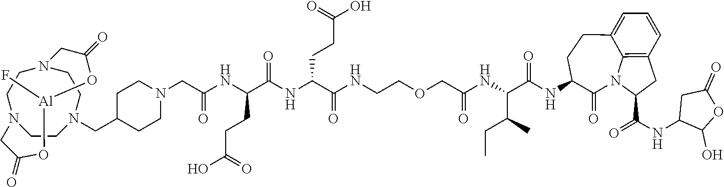 | 4-Al |
| 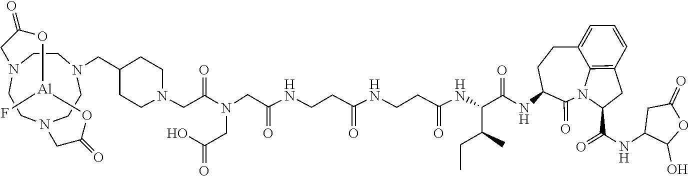 | 5-Al |
| 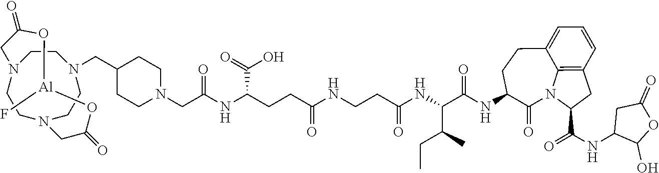 | 6-Al |
| 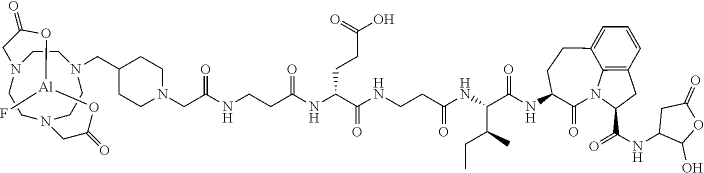 | 7-Al |

In some examples, the compound of Formula (II) is Compound 1-Al, 2-Al, 3-Al, 4-Al, 5-Al, 6-Al, or 7-Al. In one example, the compound of Formula (II) is Compound 1-Al, which may be labelled with ¹⁸F. In another example, the compound of Formula (II) is Compound 4-Al, which may be labelled with ¹⁸F. As described herein, the above examples benefit from the piperidine ring, which exhibits improved properties over other related moieties.

In other examples of Formula (IIb-B), Y is —N—, and Z is —CH₂C(O)— as in compounds of Formula (IIb-B):

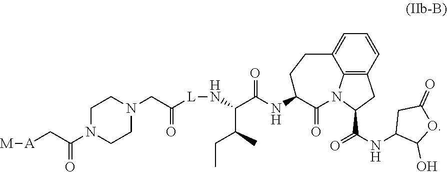

Exemplary compounds of Formula (IIb-B) include those listed in Table 6.

TABLE 6
Exemplary Compounds of Formula (IIb-B)

| Compounds | ID |
|---|---|
| 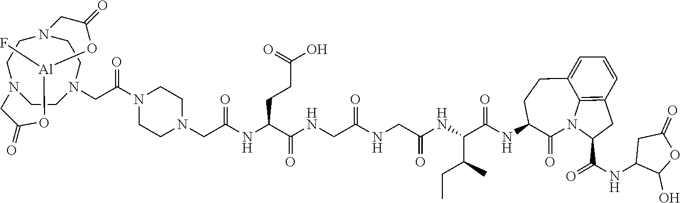 | 8-Al |
| 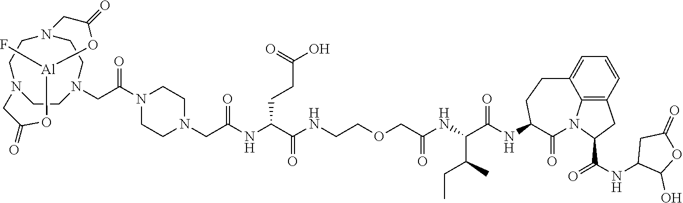 | 9-Al |
| 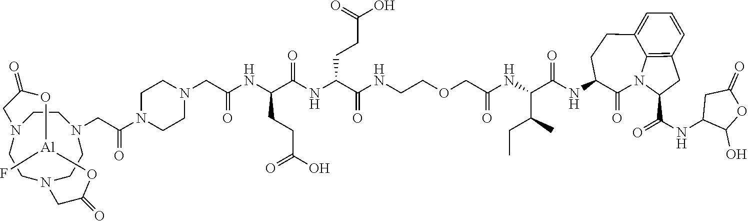 | 10-Al |
| 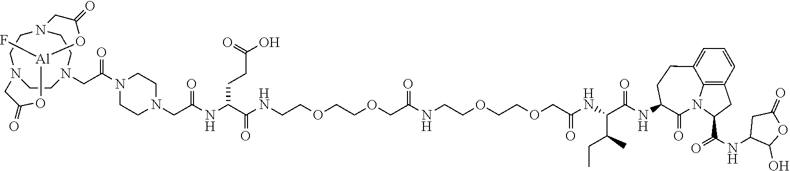 | 11-Al |
| 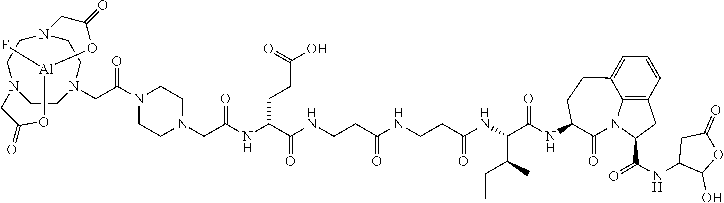 | 12-Al |
| 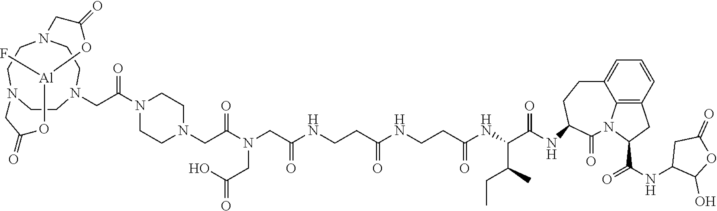 | 13-Al |
| 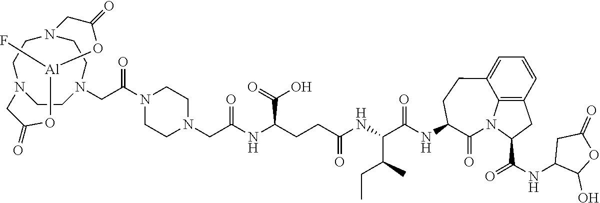 | 14-Al |
| 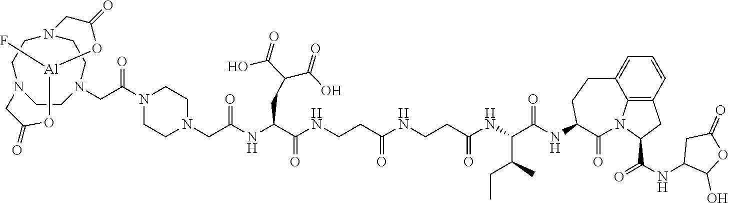 | 15-Al |
| 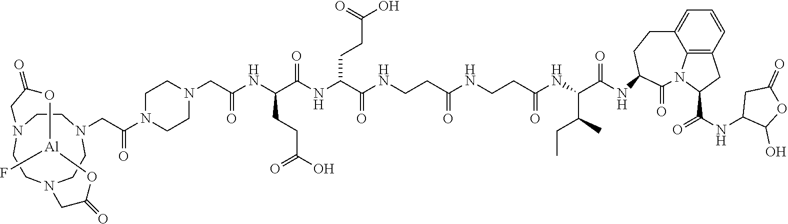 | 16-Al |
| 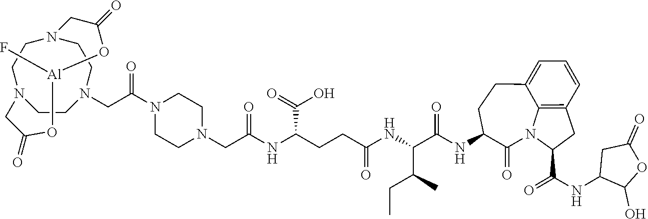 | 17-Al |

In some examples, the compound of Formula (II) is Compound 8-Al, 9-Al, 10-Al, 11-Al, 12-Al, 13-Al, 14-Al, 15-Al, 16-Al, or 17-Al. As described herein, the above examples benefit from the piperazine ring, which exhibits improved properties over many other related moieties.

As shown in Examples below, the GZB-binding compounds disclosed herein, comprising either piperidine or piperazine rings linking the peptide linker and the chelating moiety, as well as specific peptide linker structures, showed better in vivo imaging results and clearance profiles as compared with reference Compounds 18, 19, 20, and 21 which have a phenyl ring and a different L peptide linker. See International Application No.: PCT/US2021/036661, filed on Jun. 9, 2021, the relevant disclosures of which are incorporated by reference for the subject matter and purpose referenced herein.

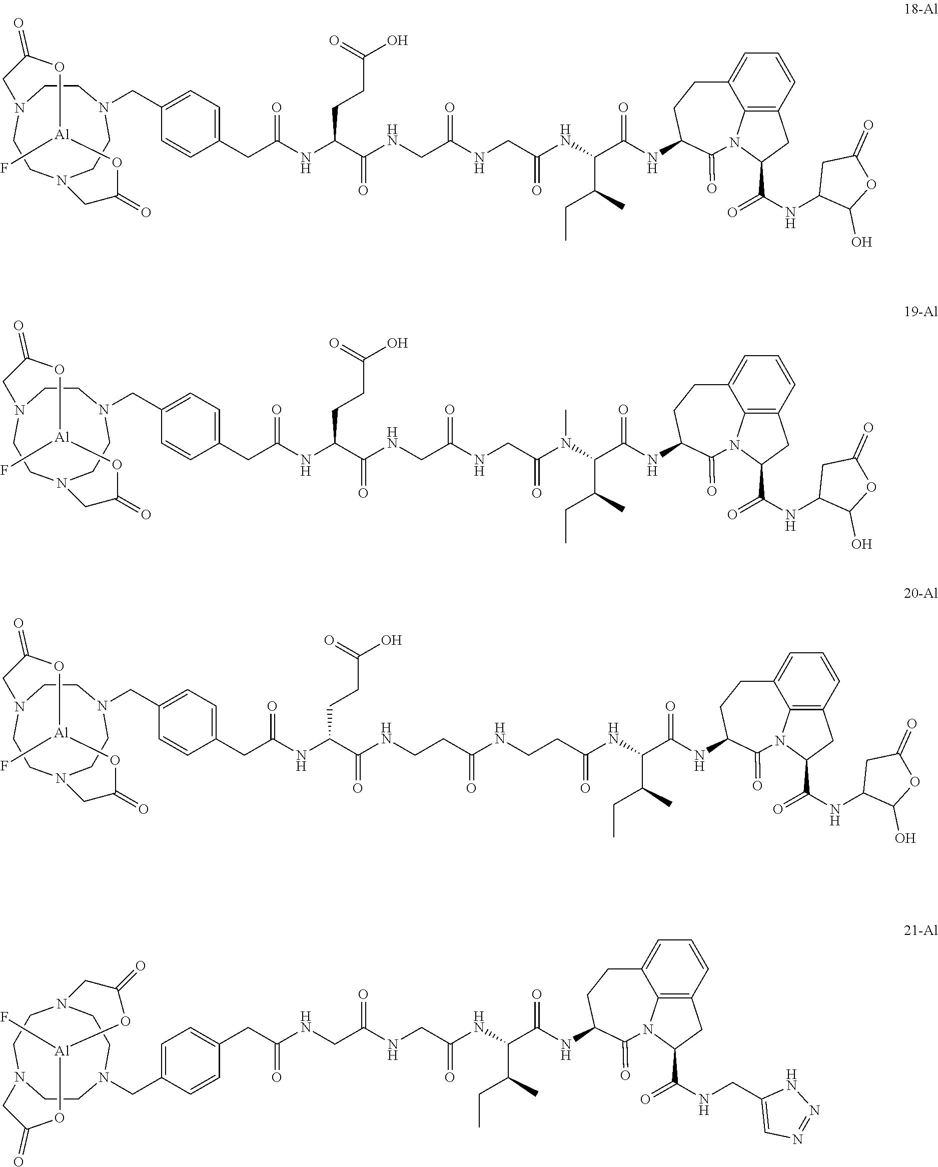

Exemplary improved properties include improved pharmacokinetics (e.g., renal clearance), pharmacodynamics, and efficacy. In particular, the improved pharmacokinetics can be seen in the absence of gut intake, the absence of radiometabolites in urine, and/or predominant renal clearance.

The above compounds, when containing a radioisotope, are useful as imaging agents in one or more of the methods provided herein. In addition, the radioisotope-containing compounds provided herein may also be useful in one or more therapeutic applications, when administered to a subject in a therapeutically effective amount. For example, the above compounds containing ¹⁸F may be useful as imaging agents (e.g., as non-toxic and/or non-therapeutic radioisotopes) when administered to the subject at low concentrations (e.g., 5 mCi). In some embodiments the isotope can be toxic. As pointed out above, the present application also includes pharmaceutically acceptable salts of the compounds described herein. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes stereoisomers of the compounds, such as stereoisomers of Compounds 1-Al and 4-Al described herein. See below. The stereoisomers result from the two chiral centers (closed ring) or one chiral center (open chain) of the hemiacetal unit as indicated by the wiggle lines:

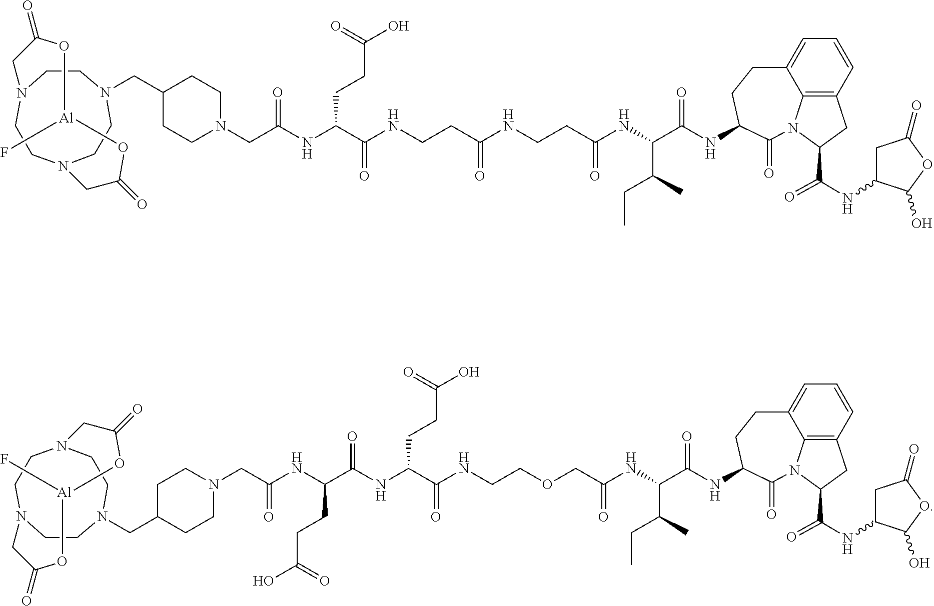

Examples of the stereoisomers of Compounds 1-Al and 4-Al are listed in Table 7 below.

TABLE 7
Stereoisomers of Compounds 1-Al and 4-Al

| Compounds | ID |
|---|---|
| 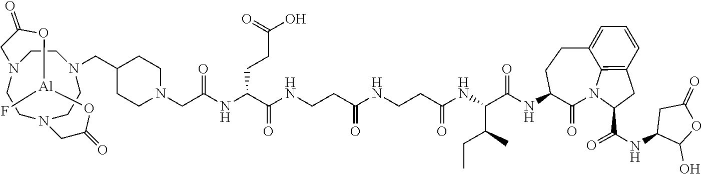 | 1-Al (S) |
| 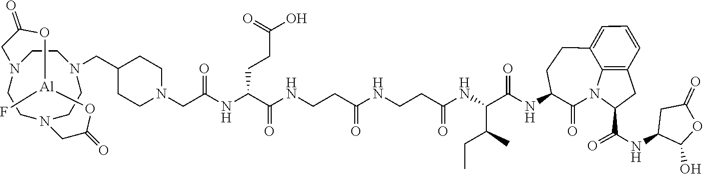 | 1-Al (S)(S) |
| 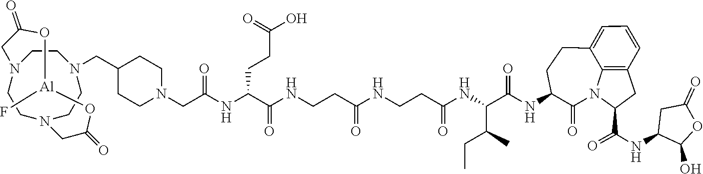 | 1-Al (S)(R) |
| 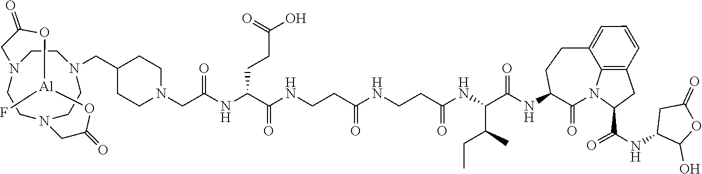 | 1-Al (R) |
| 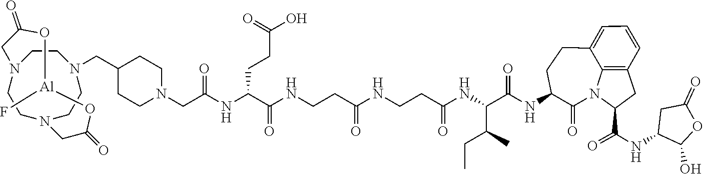 | 1-Al (R)(S) |
| 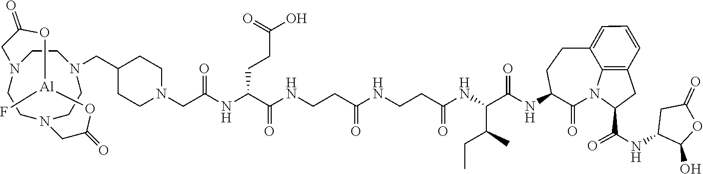 | 1-Al (R)(R) |
| 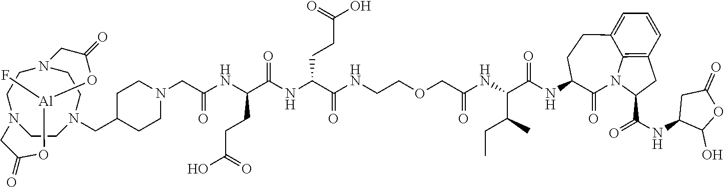 | 4-Al (S) |
| 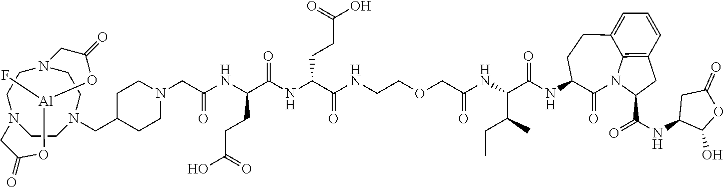 | 4-Al (S)(S) |
| 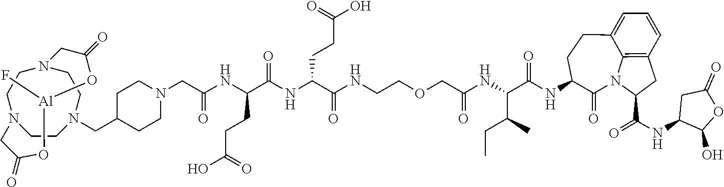 | 4-Al (S)(R) |
| 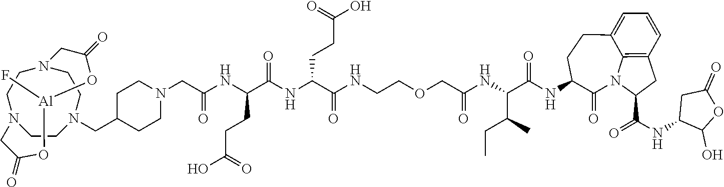 | 4-Al (R) |
| 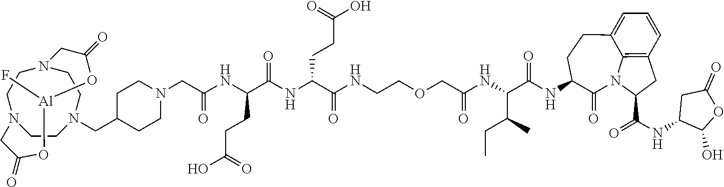 | 4-Al (R)(S) |
| 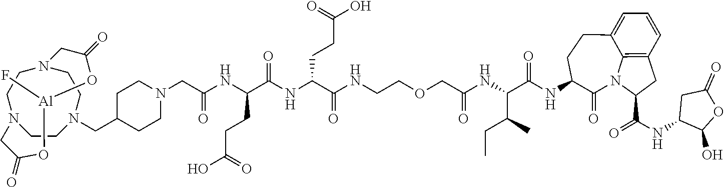 | 4-Al (R)(R) |

In specific examples, the compound for use in the present disclosure is Compound 1-Al, e.g., stereoisomer 1-Al(S).

### C. Chemical Synthesis of Granzyme B-Targeting Compounds

As will be appreciated, the compounds provided herein, including stereoisomers and salts thereof, can be prepared using known organic synthesis techniques and can be synthesized according to any of numerous possible synthetic routes.

The compounds disclosed herein, or a pharmaceutically acceptable salt thereof, can be prepared by following the exemplary protocols described below. Appropriate protective groups for use in such syntheses are known in the field. See, e.g., McOmie, Protective Groups in Organic Chemistry, (1973): 98.

General synthetic procedures, and working examples thereof, for the preparation of peptide linker L, the Fmoc-Haic(2S, 5S)—OH tricycle moiety, and the appropriate metal complexation with the chelating moiety, can be found in International Application No.: PCT/US2021/036661, filed on Jun. 9, 2021, the relevant disclosures of which are incorporated by reference for the subject matter and purpose referenced herein.

Many appropriate imaging agents (e.g., radioisotopes) are known in the art (see, for e.g., U.S. Pat. Nos. 5,021,236; 4,938,948; and 4,472,509, the disclosure of each of which is incorporated herein by reference in its entirety). Radioactively labeled compounds, or a pharmaceutically acceptable salt thereof, provided herein may be prepared according to well-known methods in the art. Synthetic methods for incorporating radioisotopes into organic compounds are well known in the art, and one of ordinary skill in the art will readily recognize other methods applicable for the compounds provided herein.

It will be appreciated by one skilled in the art that the processes described herein are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of the compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### II. Pharmaceutical Compositions

Any of the compounds of Formula (I) and Formula (II), or a pharmaceutically acceptable salt thereof, may be mixed with a pharmaceutically acceptable carrier to form a pharmaceutical composition for use in Granzyme B imaging and/or for therapeutic purposes as disclosed herein. In some embodiments, provided herein pharmaceutical compositions comprising, as the active ingredient, a compound with a metal as provided herein (a Formula (II) compound), or a pharmaceutically acceptable salt thereof, in combination with one or more pharmaceutically acceptable carriers (excipients). “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof. See Remington's Pharmaceutical Sciences, 17^{th} ed., Mack Publishing Company, Easton, Pa., 1985, p. 1418 for more information on acceptable pharmaceutical compositions.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. Parenteral administration can be in the form of a single bolus dose, or may be, for example, by a continuous perfusion pump. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution, or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

For oral administration, the composition can take the form of, for example, tablets or capsules, prepared by conventional means with acceptable excipients such as binding agents (for example, pre-gelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art.

In some embodiments, the above compounds, or a pharmaceutically acceptable salt thereof, provided herein are suitable for parenteral administration. In some embodiments, the above compounds, or a pharmaceutically acceptable salt thereof, are suitable for intravenous administration.

Pharmaceutical compositions and formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable.

In making the pharmaceutical compositions provided herein, the active ingredient is typically mixed with an excipient, diluted by an excipient, or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

Thus, the pharmaceutical compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

### III. Methods of Uses

The present application further provides a method of imaging Granzyme B using one of the above-described compounds, or a pharmaceutically acceptable salt thereof. In some embodiments, the method is an in vitro method. In some embodiments, the method is an in vivo method.

In some embodiments, the method of imaging can be performed in vitro. For example, a cell, a tissue, a cell sample, a tissue sample, may be examined to determine presence or level of Granzyme B in the cell, tissue, cell sample, or tissue sample.

Alternatively, the method of imaging as disclosed herein may be an in vivo imaging method, comprising administering the GZB-binding compound disclosed herein or a pharmaceutical composition comprising such to a subject in need thereof via a suitable route, for example, intravenous injection or local injection.

As used herein, the term “subject,” refers to any animal, including mammals and invertebrates. For example, mice, rats, other rodents, rabbits, dogs, cats, swine, cattle, sheep, horses, primates, fish, and humans. In some embodiments, the subject is a human. In some embodiments, the subject is a mouse. In some embodiments, the subject is a fish (e.g., a zebra fish).

The present application further provides a method of imaging Granzyme B in a cell or tissue, comprising:
i) contacting the cell or tissue with an effective amount of one of the above compounds, or a pharmaceutically acceptable salt thereof, and
ii) imaging the cell or tissue with a suitable imaging technique, thereby imaging Granzyme B in the cell or tissue.

The present application further provides a method of imaging Granzyme B in a sample, cell sample or tissue sample, comprising:
i) contacting the sample, cell sample or tissue sample with effective amount of one of the above compounds, or a pharmaceutically acceptable salt thereof, and
ii) imaging the sample, cell sample or tissue sample with a suitable imaging technique, thereby imaging Granzyme B in the sample, cell sample or tissue sample.

As used herein within the context of imaging, the term “sample” refers to a biological sample other than cell or tissue sample which is obtained from a subject. For example, the sample includes but not limited to saliva, blood, and urine.

The present application further provides a method of imaging Granzyme B in a subject, comprising:
i) administering to the subject an effective amount of one of the above compounds, or a pharmaceutically acceptable salt thereof, and
ii) imaging the subject with a suitable imaging technique, thereby imaging Granzyme B in the subject.

The present application further provides a method of imaging an immune response in a cell or tissue sample, comprising:
i) contacting the cell or tissue sample with an effective amount of one of the above compounds, or a pharmaceutically acceptable salt thereof, and
ii) imaging the cell or tissue sample with a suitable imaging technique, thereby imaging the immune response in the cell or tissue sample.

The present application further provides a method of imaging an immune response in a subject, comprising:
i) administering to the subject an effective amount of one of the above compounds, or a pharmaceutically acceptable salt thereof, and
ii) imaging the subject with a suitable imaging technique, thereby imaging the immune response in the subject.

The present application further provides a method of monitoring treatment of a disease in a subject, comprising:
i) administering to the subject an effective amount of one of the above compounds, or a pharmaceutically acceptable salt thereof, and
ii) imaging the subject with a suitable imaging technique.

The present application further provides a method of monitoring an immune response in the treatment of a disease in a subject, comprising:
i) administering to the subject an effective amount of one of the above compounds, or a pharmaceutically acceptable salt thereof, and
ii) imaging the subject with a suitable imaging technique.

In some embodiments, the methods provided herein further comprise waiting a time sufficient to allow the compound, or a pharmaceutically acceptable salt thereof, to accumulate at a cell or tissue site (e.g., a cell or tissue site in a subject) associated with the disease, prior to imaging.

In some embodiments, the methods provided herein further comprise waiting a time sufficient to allow the compound, or a pharmaceutically acceptable salt thereof, to bind Granzyme B at a cell or tissue site (e.g., a cell or tissue site in a subject) associated with the disease, prior to imaging.

In some embodiments, the time sufficient is from about 30 seconds to about 24 hours, for example, about 30 seconds to about 24 hours, about 30 seconds to about 12 hours, about 30 seconds to about 6 hours, about 30 seconds to about 2 hours, about 30 seconds to about 1 hour, about 30 seconds to about 30 minutes, about 30 seconds to about 10 minutes, about 10 minutes to about 24 hours, about 10 minutes to about 12 hours, about 10 minutes to about 6 hours, about 10 minutes to about 2 hours, about 10 minutes to about 1 hour, about 10 minutes to about 30 minutes, about 30 minutes to about 24 hours, about 30 minutes to about 12 hours, about 30 minutes to about 6 hours, about 30 minutes to about 2 hours, about 30 minutes to about 1 hour, about 1 hour to about 24 hours, about 1 hour to about 12 hours, about 1 hour to about 6 hours, about 1 hour to about 2 hours, about 2 hours to about 24 hours, about 2 hours to about 12 hours, about 2 hours to about 6 hours, about 6 hours to about 24 hours, about 6 hours to about 12 hours, or about 12 hours to about 24 hours.

In some embodiments, the suitable imaging technique is a non-invasive imaging technique. In some embodiments, the suitable imaging technique is a minimally invasive imaging technique. As used herein, the term “minimally invasive imaging technique” comprises imaging techniques employing the use of an internal probe or injection of one of the above compounds, or a pharmaceutically acceptable salt thereof, or radiotracer via syringe.

Exemplary imaging techniques include, but are not limited to, fluoroscopic imaging, x-ray imaging, magnetic resonance imaging (MRI), ultrasound imaging, photoacoustic imaging, thermographic imaging, tomographic imaging, echocardiographic imaging, positron emission tomography (PET) imaging, PET with computed tomography (CT) imaging, PET-MRI, single-photon emission computed tomography (SPECT), and ultrasound imaging. In some embodiments, the suitable imaging technique is selected from the group consisting of PET imaging, PET-CT, PET-MRI, and SPECT.

In some embodiments, the suitable imaging technique is selected from the group consisting of PET imaging, PET with computed tomography imaging, and PET with magnetic resonance imaging (MRI). In some embodiments, the suitable imaging technique is selected PET imaging.

The results from any of the Granzyme B imaging methods disclosed herein may be relied on for diagnostic and/or prognostic purposes. In some embodiments, the results can be relied on to identify a patient suitable for treatment of an immunoregulatory abnormality (disease) with a therapeutic agent (e.g., an anti-inflammatory agent, steroids, immunotherapy agents, chemotherapeutic agents, or therapeutic antibodies). In other embodiments, the results can be relied on for monitoring efficacy of a therapeutic agent such as those provided herein. For example, presence of Granzyme B or an increase of the GZB level may indicate that the patient is suitable and/or responsive to the therapeutic agent. In that case, the method disclosed herein may further comprise administering the therapeutic agent to the patient for treating the target disease.

In some embodiments, a disease as described herein is selected from the group consisting of an autoimmune disorder, an inflammatory disorder, a skin disorder, cancer, and a cardiovascular disorder. As used herein, the term “disease” is used interchangeable with the term “immunoregulatory abnormality.”

In some embodiments, the disease is a cancer. In some embodiments, the cancer comprises a solid tumor. In some embodiments, the cancer is a hematological cancer (e.g., leukemia, lymphoma, and the like). Exemplary solid cancers include, but are not limited to, brain, breast cancer, cervical cancer, colorectal cancer, lung cancer, lymphoma, melanoma, bladder cancer, renal cell carcinoma, multiple myeloma, pancreatic cancer, and prostate cancer. Exemplary hematological cancers include, but are not limited to, Hairy-cell leukemia, Kaposi's sarcoma, follicular lymphoma, chronic myeloid leukemia, cutaneous T-cell lymphoma, peripheral T-cell lymphoma, T-cell prolymphocytic leukemia, Classical Hodgkin's lymphoma, B-cell non-Hodgkin's lymphoma, chronic lymphocytic leukemia, acute myeloid leukemia, myelodysplastic syndrome, primary myelofibrosis, post-essential thrombocythemia myelofibrosis, post-polycythemia vera myelofibrosis. Other examples include melanoma, renal cell carcinoma, prostate cancer, non-small cell lung cancer, small cell lung cancer, glioblastoma, hepatocellular carcinoma, urothelial carcinoma, esophageal carcinoma, gastroesophageal carcinoma, gastric cancer, multiple myeloma, colon cancer, rectal cancer, squamous cell carcinoma of the head and neck, epithelial ovarian cancer (EOC), primary peritoneal cancer, fallopian tube carcinoma, HER2+ breast cancer, ER+/PR+/HER2-breast cancer, triple-negative breast cancer, gastric cancer, pancreatic cancer, bladder cancer, Merkel cell cancer, nasopharyngeal cancer, adrenocortical carcinoma, meningioma, neuroblastoma, retinoblastoma, osteosarcoma, rhabdomyosarcoma, Ewing's sarcoma, liposarcoma, fibrosarcoma, leiomyosarcoma, peripheral primitive neuroectodermal tumor, squamous cell carcinoma of the cervix, squamous cell carcinoma of the vagina, and squamous cell carcinoma of the vulva. In some examples, the cancer is colon cancer.

Additional exemplary immunoregulatory abnormalities include, but are not limited to, graft-versus-host disease, rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, rheumatic fever, post-infectious glomerulonephritis, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenia purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia, alopecia senilis by preventing epilation, alopecia senilis by providing hair germination and/or promoting hair generation and hair growth, muscular dystrophy, pyoderma, Sezary's syndrome, Addison's disease, ischemia-reperfusion injury of organs, transplantation disease, ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, aging, carcinogenesis, metastasis of carcinoma and hypobaropathy, histamine or leukotriene-C4 release associated diseases, Behcet's disease, autoimmune hepatitis, primary biliary cirrhosis, sclerosing cholangitis, partial liver resection, acute liver necrosis, necrosis caused by toxin, viral hepatitis, shock, anoxia, B-virus hepatitis, non-A/non-B hepatitis, cirrhosis, alcoholic cirrhosis, hepatic failure, fulminant hepatic failure, late-onset hepatic failure, acute-on-chronic liver failure, cytomegalovirus infection, HCMV infection, AIDS, senile dementia, trauma, chronic bacterial infection, malignancy of lymphoid origin, acute lymphocytic leukemia, chronic lymphocytic leukemia, acute lymphocytic lymphoma, and chronic lymphocytic lymphoma.

Exemplary autoimmune diseases include, but are not limited to, systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

In some embodiments, the disease can be bone marrow rejection, organ transplant rejection, and graft-versus-host disease.

When employed in methods of treating a disease, the above compounds of Formula (II), or pharmaceutically acceptable salts thereof, provided herein can be administered in combination with one or more additional therapeutic agents. In some instances, the additional therapeutic agent induces an immune response in a subject receiving the treatment. The Formula (II) compounds can be used to monitor such an immune response based on presence/absence of GZB or change of the GZB levels in the subject.

In some embodiments, a compound of Formula (II) may be given to a patient after the patient receives at least one dose of the additional therapeutic agent. Based on the GZB-imaging results arising from the compound of Formula (II) compound, the patient may continue the treatment comprising the additional therapeutic agent. In some examples, the dosing and/or dosing schedule of the additional therapeutic agent may be adjusted.

Examples of the additional therapeutic agents include, but are not limited to, anti-inflammatory agents, steroids, immunotherapy agents, chemotherapeutic agents, and therapeutic antibodies.

In some embodiments, the therapeutic agent is an antibody. Exemplary antibodies for use in a combination therapy include, but are not limited to, trastuzumab (e.g. anti-HER2), ranibizumab (e.g. anti-VEGF-A), bevacizumab (e.g. anti-VEGF), panitumumab (e.g. anti-EGFR), cetuximab (e.g. anti-EGFR), rituxan (anti-CD20), antibodies directed to c-MET, and antibody inhibitors of Granzyme B (e.g., Clone GB11, Clone GrB-7, and NCL-L-Gran-B), ipilimumab (anti-CTLA-4), nivolumab (anti-PD-1), pembrolizumab (anti-PD-1), atezolizumab (anti-PD-1), elotuzumab (anti-SLAM7), and daratumumab (anti-CD38).

In some embodiments, the additional therapeutic agent is a steroid. Exemplary steroids include corticosteroids such as cortisone, dexamethasone, hydrocortisone, methylprednisolone, prednisolone, and prednisone. In some embodiments, the additional therapeutic agent is a corticosteroid.

In some embodiments, the additional therapeutic agent is an anti-inflammatory compound. Exemplary anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

In some embodiments, the additional therapeutic agent is chemotherapeutic agent. Exemplary chemotherapeutic agents include, but are not limited to, a cytostatic agent, cisplatin, doxorubicin, taxol, etoposide, irinotecan, topotecan, paclitaxel, docetaxel, epothilones, tamoxifen, 5-fluorouracil, methotrexate, temozolomide, cyclophosphamide, SCH 66336, R115777, L778,123, BMS 214662, gefitinib, erlotinib hydrochloride, antibodies to EGFR, imatinib mesylate, intron, ara-C, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, vinorelbine, porfimer, erbitux, liposomal, thiotepa, altretamine, melphalan, trastuzumab, fulvestrant, exemestane, ifosfamide, rituximab, C225, alemtuzumab, clofarabine, cladribine, aphidicolin, sunitinib, dasatinib, tezacitabine, Sml1, triapine, didox, trimidox, amidox, 3-AP, MDL-101,731, bendamustine, ofatumumab, and GS-1101 (also known as CAL-101).

In some embodiments, the chemotherapeutic agent is selected from the group consisting of an alkylating agent (e.g., busulfan, chlorambucil, cisplatin, cyclophosphamide (cytoxan), dacarbazine, ifosfamide, mechlorethamine (mustargen), and melphalan), a nitrosourea (e.g., carmustine, lomustine, semustine, and streptozocin), a triazine (e.g., dacarbazine) an anti-metabolite (e.g., 5-fluorouracil (5-FU), cytarabine (Ara-C), fludarabine, gemcitabine, and methotrexate), a purine analog (e.g., 6-mercaptopurine, 6-thioguanine, and pentostatin (2-deoxycoformycin)), a mitotic inhibitor (e.g., docetaxel, etoposide (VP16), teniposide, paclitaxel, taxol, vinblastine, vincristine, and vinorelbine), an anti-tumor antibiotic (e.g., bleomycin, dactinomycin, daunorubicin, doxorubicin, mitomycin, plicamycin, and idarubicin), a platinum chemotherapeutic agent (e.g., cisplatin and carboplatin), an anthracenedione (e.g., mitoxantrone), a toxin (e.g., ricin A-chain (Burbage, Leukemia research, 21.7 (1997): 681-690), diphtheria toxin A (Massuda et al., Proceedings of the National Academy of Sciences, 94.26 (1997): 14701-14706; Lidor, American journal of obstetrics and gynecology, 177.3 (1997): 579-585), pertussis toxin A subunit, E. colienterotoxin toxin A subunit, cholera toxin A subunit and Pseudomonas toxin c-terminal), and a gene therapy vector (e.g., a signal transducing protein (e.g., Src, Abl, and Ras), Jun, Fos, and Myc).

In some embodiments, the additional therapeutic agent is an immunotherapeutic agent. An immunotherapeutic agent generally triggers immune effector cells and molecules to target and destroy cells (e.g., cancer cells). The immune effector may be, for example, an antibody specific for a marker on the surface of a cell (e.g. a tumor cell). The antibody alone may serve as an effector of therapy or it may recruit other cells to effect cell killing. Various effector cells include, but are not limited to, cytotoxic T cells and NK cells.

Exemplary immunotherapeutic agents include, but are not limited to, azathioprine, chlorambucil, cyclophosphamide, cyclosporine, daclizumab, infliximab, methotrexate, tacrolimus, immune stimulators (e.g., IL-2, IL-4, IL-12, GM-CSF, tumor necrosis factor; interferons alpha, beta, and gamma; F42K and other cytokine analogs; a chemokine such as MIP-1, MIP-1β, MCP-1, RANTES, IL-8; or a growth factor such FLT3 ligand), an antigenic peptide, polypeptide or protein, or an autologous or allogenic tumor cell composition (see e.g., Ravindranath & Morton, International reviews of immunology, 7.4 (1991): 303-329), hormonal therapy, adrenocorticosteroids, progestins (e.g., hydroxyprogesterone caproate, medroxyprogesterone acetate, and megestrol acetate), estrogens (e.g., diethylstilbestrol and ethinyl estradiol), anti-estrogens (e.g., testosterone propionate and fluoxymesterone), anti-androgens (e.g., flutamide), and gonadotropin-releasing hormone analogs (e.g., leuprolide). Additional immunotherapeutic agents are known in the art, and can be found, for example, in Rosenberg et al, New England Journal of Medicine, 319.25 (1988): 1676-1680; and Rosenberg et al, Annals of surgery, 210.4 (1989): 474).

The therapeutic agents provided herein can be effective over a wide dosage range and are generally administered in an effective amount. It will be understood, however, that the amount of the therapeutic agent actually administered will usually be determined by a physician, according to the relevant circumstances, including the condition to be imaged, the chosen route of administration, the actual compound administered, the age, weight, and response of the individual subject, the severity of the subject's symptoms, and the like.

Additional information can be found in International Application No.: PCT/US2021/036661, filed on Jun. 9, 2021, the relevant disclosures of which are incorporated by reference for the subject matter and purpose referenced herein.

### IV. Kits for Granzyme B Imaging

Also encompassed by the disclosure are kits (e.g., pharmaceutical packs) for Granzyme B imaging, using any of the GZB-binding compounds disclosed herein. The kits provided may comprise a container (e.g., a vial, ampule, bottle, syringe, and/or dispenser package, or other suitable container), in which a pharmaceutical composition as disclosed herein may be placed. In some embodiments, provided kits may optionally further include a second container comprising a pharmaceutical excipient for dilution or suspension of a pharmaceutical composition. In some embodiments, the pharmaceutical composition provided in the first container and the second container are combined to form one unit dosage form. In some embodiments, the kit may comprise additional containers comprising one or more additional therapeutic agents as disclosed herein, for example, anti-inflammatory agents, steroids, immunotherapy agents, chemotherapeutic agents, and therapeutic antibodies as described herein.

In certain embodiments, a kit described herein further includes instructions for using the compounds or composition included in the kit. A kit described herein may also include information as required by a regulatory agency such as the U.S. Food and Drug Administration (FDA). In certain embodiments, the information included in the kits is prescribing information. In certain embodiments, the kits and instructions provide for imaging Granzyme B and for assessing treatment efficacy by any of the therapeutic agents disclosed herein in a subject in need thereof. A kit described herein may include one or more additional pharmaceutical agents described herein as a separate composition.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Synthesis of Exemplary Compounds of Formula (I)

### Preparation of Chelating Moiety Coupling Partner

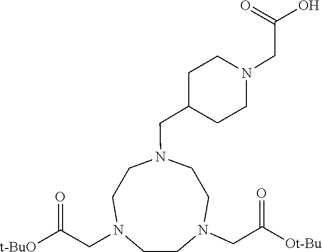 [SMILES: CC(C)(C)OC(=O)CN1CCN(CC(=O)OC(C)(C)C)CCN(CC2CCN(CC(=O)O)CC2)CC1]

### 2-(4-((4,7-bis(2-(tert-butoxy)-2-oxoethyl)-1,4,7-triazonan-1-yl)methyl)piperidin-1-yl)acetic Acid

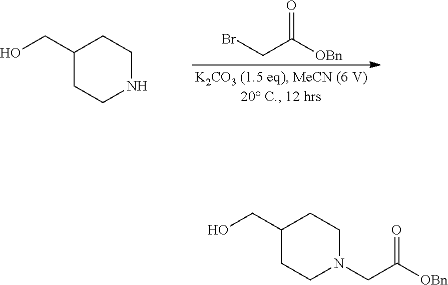 [SMILES: OCC1CCNCC1 ; O=C(CBr)OCc1ccccc1 ; O=C(CN1CCC(CO)CC1)OCc1ccccc1]

Step 1: To a solution of piperidin-4-ylmethanol (90.0 g, 781 mmol) in MeCN (540 mL) was added benzyl 2-bromoacetate (179 g, 781 mmol, 123 mL) and K₂CO₃ (162 g, 1.17 mol). The mixture was stirred at 20° C. for 12 hrs. TLC (petroleum ether:ethyl acetate=0:1, R_{f}=0.20) showed the starting material was consumed completely. The mixture was filtered, and the filtrate was concentrated in vacuum. The residue was purified by column chromatography (SiO₂, petroleum ether:ethyl acetate=50:1 to 0:1). Benzyl 2-(4-(hydroxymethyl)piperidin-1-yl)acetate (92.0 g, 349 mmol) was obtained as yellow oil.

¹H NMR: (400 MHZ, CDCl₃) δ 7.35-7.37 (m, 5H), 5.16 (s, 2H), 3.49 (d, J=6.4 Hz, 2H), 3.26 (s, 2H), 2.95 (d, J=11.2 Hz, 2H), 2.16-2.22 (m, 2H), 1.72 (d, J=13.2 Hz, 2H), 1.59 (s, 1H), 1.49-1.55 (m, 1H), 1.30-1.40 (m, 2H).

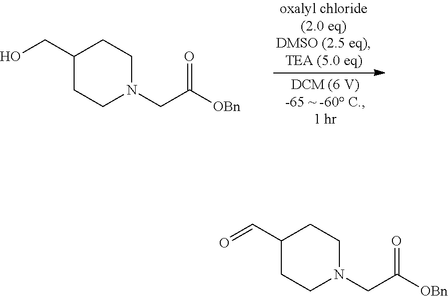 [SMILES: O=C(CN1CCC(CO)CC1)OCc1ccccc1 ; O=CC1CCN(CC(=O)OCc2ccccc2)CC1]

Step 2: To a solution of oxalyl chloride (88.7 g, 699 mmol, 61.2 mL) in DCM (460 mL) was added DMSO (68.2 g, 873 mmol, 68.2 mL) dropwise at −65° C. and the mixture was stirred at −65° C. for 30 mins. A solution of benzyl 2-(4-(hydroxymethyl)piperidin-1-yl)acetate (92.0 g, 349 mmol) in DCM (92.0 mL) was added dropwise below −60° C., followed by TEA (177 g, 1.75 mol, 243 mL). The resulting mixture was stirred at −65° C. for another 30 min. TLC (dichloromethane:methanol=10:1, R^{f}=0.33) showed the starting material was consumed completely. The reaction mixture was quenched by addition NaHCO₃ (500 mL) at 0° C., and then extracted once with DCM (100 mL). The combined organic layers were washed with once brine (100 mL), dried over anhydrous Na₂SO₄, filtered, and concentrated in vacuum. Benzyl 2-(4-formylpiperidin-1-yl)acetate (135 g, crude) was obtained as yellow oil.

¹H NMR: 400 MHz, CDCl₃ δ 9.65 (d, J=1.2 Hz, 1H), 7.33-7.38 (m, 5H), 5.17 (s, 2H), 3.29 (s, 2H), 2.86-2.91 (m, 2H), 2.34-2.40 (m, 2H), 2.19-2.28 (m, 1H), 1.90-1.95 (m, 2H), 1.70-1.80 (m, 2H).

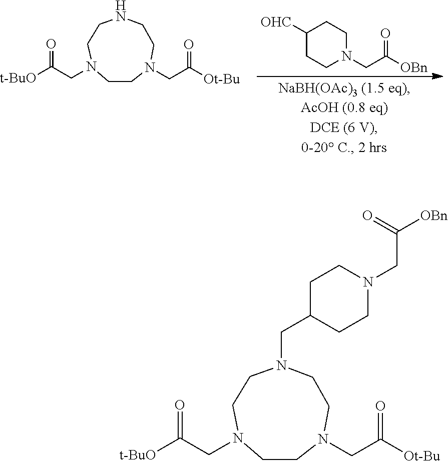 [SMILES: CC(C)(C)OC(=O)CN1CCNCCN(CC(=O)OC(C)(C)C)CC1 ; O=CC1CCN(CC(=O)OCc2ccccc2)CC1 ; CC(C)(C)OC(=O)CN1CCN(CC(=O)OC(C)(C)C)CCN(CC2CCN(CC(=O)OCc3ccccc3)CC2)CC1]

Step 3: A mixture of benzyl 2-(4-formylpiperidin-1-yl)acetate (28.1 g, 107 mmol) in DCE (192 mL) was added di-tert-butyl 2,2′-(1,4,7-triazonane-1,4-diyl)diacetate (32.0 g, 89.5 mmol) and AcOH (4.30 g, 71.6 mmol, 4.10 mL) at 0° C. The mixture was stirred for 1 hr at 0° C. Then NaBH(OAc)₃ (28.5 g, 134 mmol, 1.50 eq) was added in portions. The mixture was stirred at 20° C. for 1 hr. The reaction mixture was quenched by addition NaHCO₃ (200 mL), and then extracted five times with DCM (50.0 mL). The combined organic layers were washed with once brine (50.0 mL), dried over Na₂SO₄, filtered, and concentrated under reduced pressure to give a residue. The crude product from another two reaction, starting from 30.0 g di-tert-butyl 2,2′-(1,4,7-triazonane-1,4-diyl)diacetate and from 32.0 g di-tert-butyl 2,2′-(1,4,7-triazonane-1,4-diyl)diacetate, respectively, were combined for further purification. The combined crude product was triturated with EtOAc (120 mL) for 12 hrs to afford 51.0 g of di-tert-butyl 2,2′-(7-((1-(2-(benzyloxy)-2-oxoethyl)piperidin-4-yl)methyl)-1,4,7-triazonane-1,4-diyl)diacetate as white solid.

ES/MS m/z 603.5 (M+H)⁺.

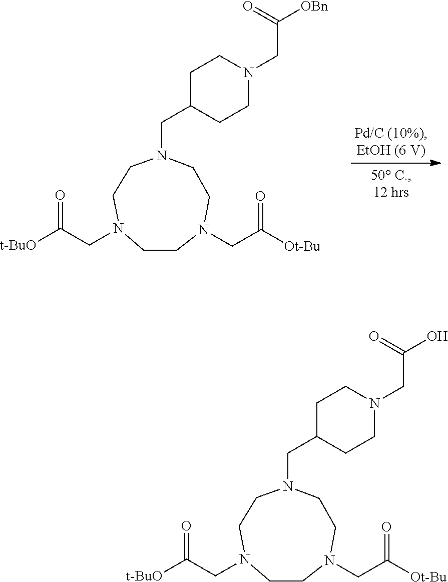 [SMILES: CC(C)(C)OC(=O)CN1CCN(CC(=O)OC(C)(C)C)CCN(CC2CCN(CC(=O)OCc3ccccc3)CC2)CC1 ; CC(C)(C)OC(=O)CN1CCN(CC(=O)OC(C)(C)C)CCN(CC2CCN(CC(=O)O)CC2)CC1]

Step 4: To a solution of di-tert-butyl 2,2′-(7-((1-(2-(benzyloxy)-2-oxoethyl)piperidin-4-yl)methyl)-1,4,7-triazonane-1,4-diyl)diacetate (51.0 g, 84.6 mmol) in EtOH (306 mL) was added Pd/C (5.10 g, 10% purity) under N₂ atmosphere. The suspension was degassed and purged with H₂ for 3 times. The mixture was stirred under H₂ (50 Psi) at 50° C. for 4 hrs. The mixture was filtered through celite, and the filtrate was concentrated in vacuum. The crude product was triturated with MTBE (150 mL) for 4 hrs. 2-(4-((4,7-bis(2-(tert-butoxy)-2-oxoethyl)-1,4,7-triazonan-1-yl)methyl)piperidin-1-yl) acetic acid (30.0 g, 56.9 mmol) was obtained as off-white foam.

ES/MS m/z 513.4 (M+H)+.

### General Peptide Synthesis Procedure:

Peptides were synthesized following standard Fmoc solid-phase peptide synthesis procedures using H-Asp(OtBu)-H resin. Final peptides were deprotected and cleaved from the resin following a two-step procedure: 1) treatment with either trifluoroacetic acid (TFA) at room temperature for 2 h or TFA/dichloromethane (DCM) at room temperature overnight, then concentrated; 2) treatment with 0.1% TFA in acetonitrile/water (60:40) at 60° C. for 1 h (https://www.emdmillipore.com/US/en/product/H-AspOtBu-H-NovaSyn-TG-resin, MDA_CHEM-856072 #documentation). Crude peptides were either concentrated or lyophilized, then subjected to preparative HPLC purification (0.1% formic acid or 0.1% TFA in water/acetonitrile mobile phase). Product-containing fractions were collected and lyophilized to afford peptides as white, fluffy solids.

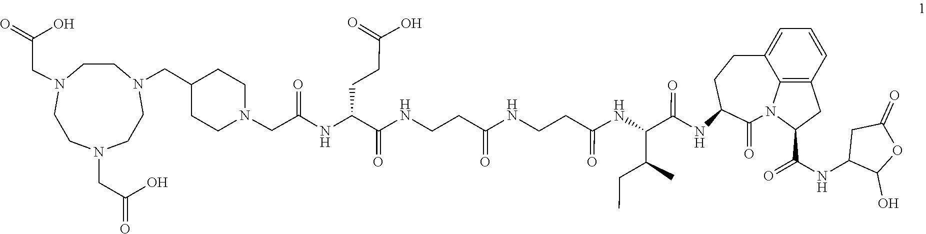 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (36 mg) was prepared.

ES/MS m/z 1112.6 (M+H)⁺.

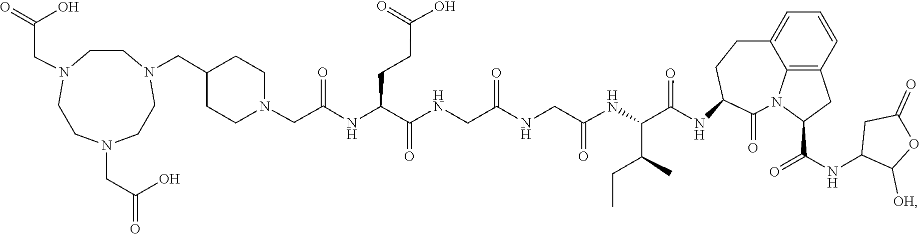 [SMILES: CC[C@H](C)[C@H](NC(=O)CNC(=O)CNC(=O)[C@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperidin-4-yl)methyl)-1,4,7-triazonane-1,4-diyl)diacetic acid (100 mg) was obtained.

ES/MS m/z 1084.5 (M+H)⁺.

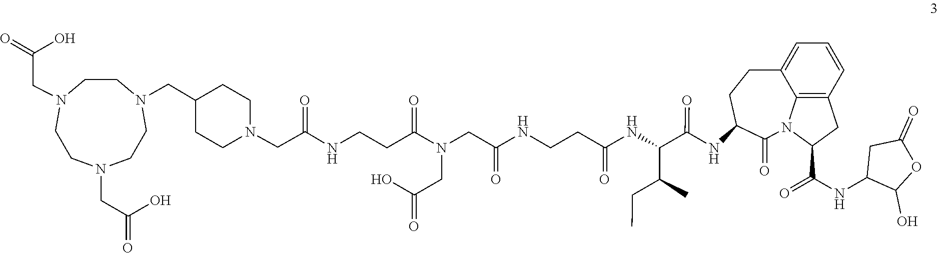 [SMILES: CC[C@H](C)[C@H](NC(=O)CCNC(=O)CN(CC(=O)O)C(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((15S,16S)-7-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (49 mg) was prepared.

ES/MS m/z 1098.4 (M+H)⁺.

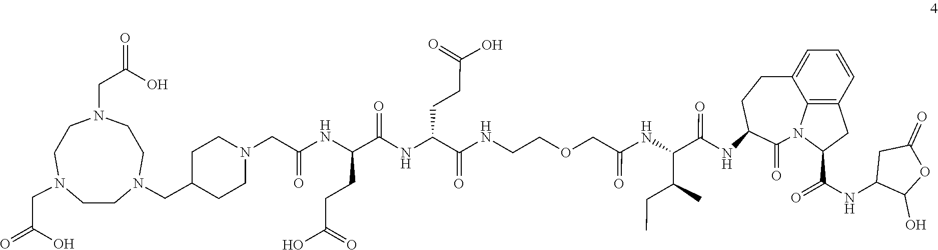 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (80 mg) was obtained.

ES/MS m/z 1200.6 (M+H)⁺.

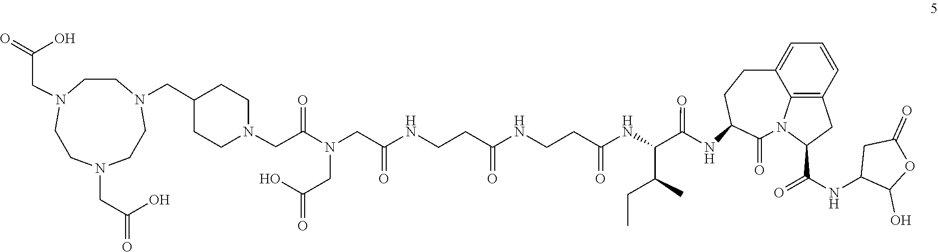 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (28 mg) was obtained.

ES/MS m/z 1098.6 (M+H)⁺.

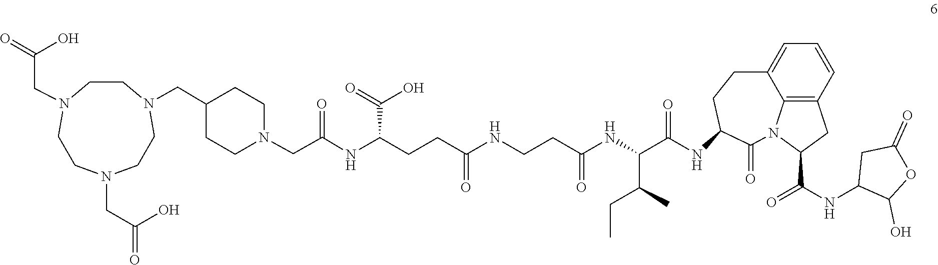 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-((1-(2-(((1S)-1-carboxy-4-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid (32 mg) was obtained.

ES/MS m/z 1041.6 (M+H)⁺.

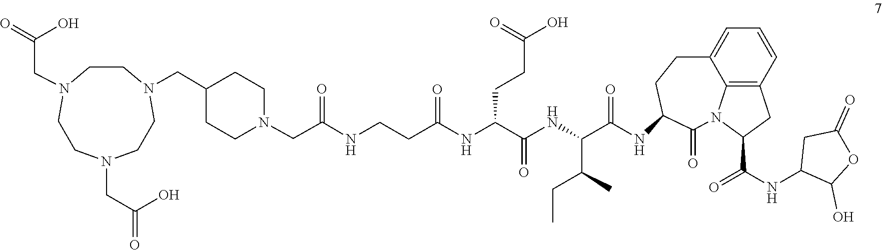 [SMILES: CC[C@H](C)[C@H](NC(=O)[C@@H](CCC(=O)O)NC(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((8R,15S,16S)-8-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (24.6 mg) was obtained.

ES/MS m/z 1112.5 (M+H)⁺.

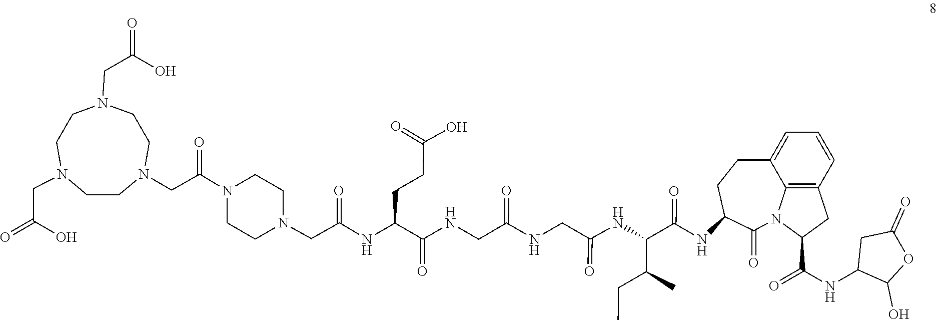 [SMILES: CC[C@H](C)[C@H](NC(=O)CNC(=O)CNC(=O)[C@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperazin-1-yl)-2-oxoethyl)-1,4,7-triazonane-1,4-diyl)diacetic acid (100 mg) was obtained.

ES/MS m/z 1113.5 (M+H)⁺.

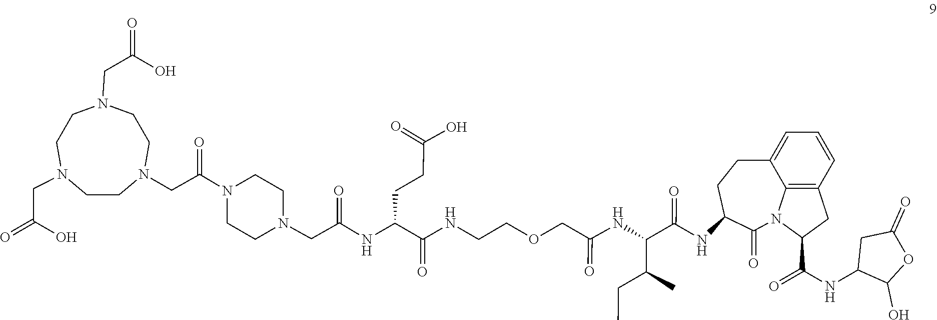 [SMILES: CC[C@H](C)[C@H](NC(=O)COCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,11-trioxo-9-oxa-3,6,12-triazahexadecyl)piperazin-1-yl)-2-oxoethyl)-1,4,7-triazonane-1,4-diyl)diacetic acid (78 mg) was obtained.

ES/MS m/z 1100.5 (M+H)⁺.

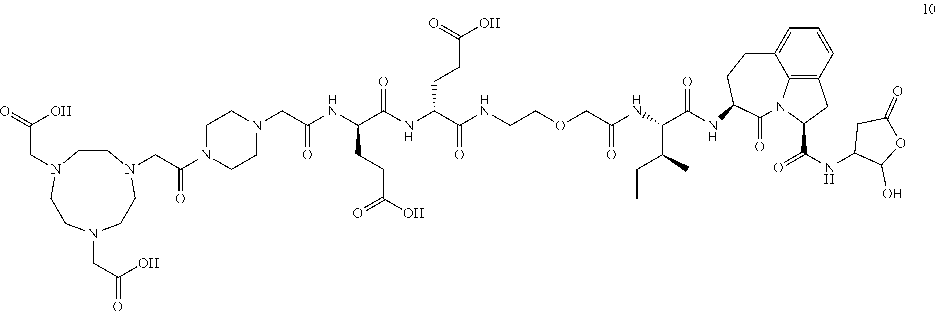 [SMILES: CC[C@H](C)[C@H](NC(=O)COCCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperazin-1-yl)-2-oxoethyl)-1,4,7-triazonane-1,4-diyl)diacetic acid (22 mg) was obtained.

ES/MS m/z 1229.7 (M+H)⁺.

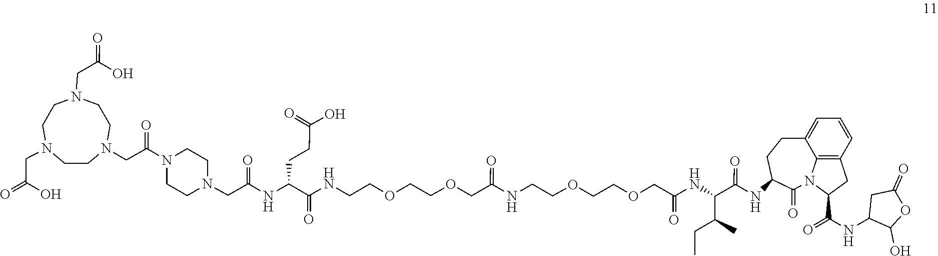 [SMILES: CC[C@H](C)[C@H](NC(=O)COCCOCCNC(=O)COCCOCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,25S,26S)-4-(2-carboxyethyl)-25-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-26-methyl-2,5,14,23-tetraoxo-9,12,18,21-tetraoxa-3,6,15,24-tetraazaoctacosyl)piperazin-1-yl)-2-oxoethyl)-1,4,7-triazonane-1,4-diyl)diacetic acid (46 mg) was obtained.

ES/MS m/z 1289.6 (M+H)+

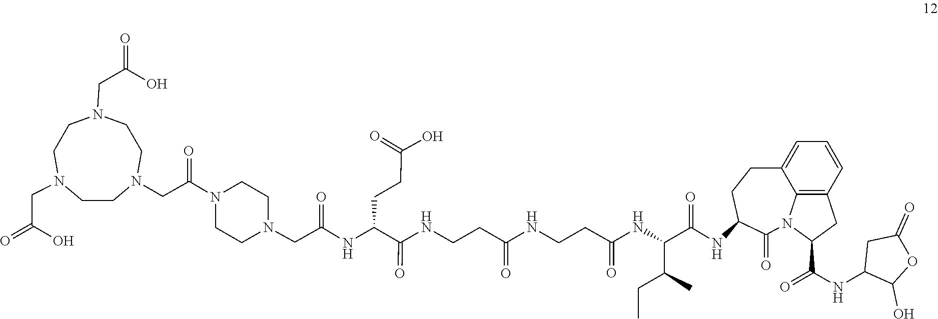 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (30 mg) was obtained.

ES/MS m/z 1141.6 (M+H)⁺.

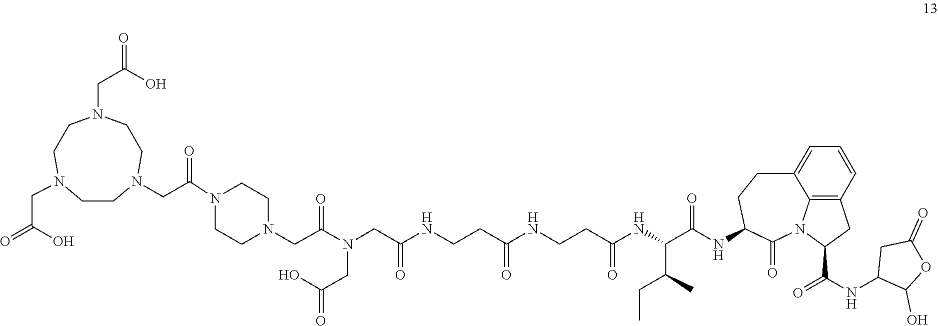 [SMILES: CC[C@H](C)[C@H](NC(=O)CCNC(=O)CCNC(=O)CN(CC(=O)O)C(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (29 mg) was obtained.

ES/MS m/z 1127.6 (M+H)⁺.

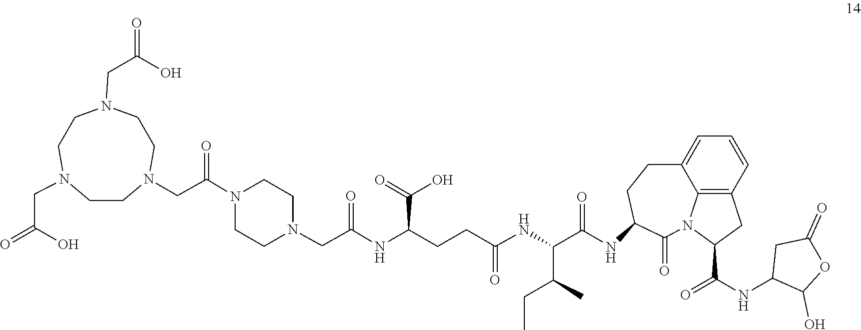 [SMILES: CC[C@H](C)[C@H](NC(=O)CC[C@@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-(2-(4-(2-(((1R)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (32 mg) was obtained.

ES/MS m/z 999.5 (M+H)⁺.

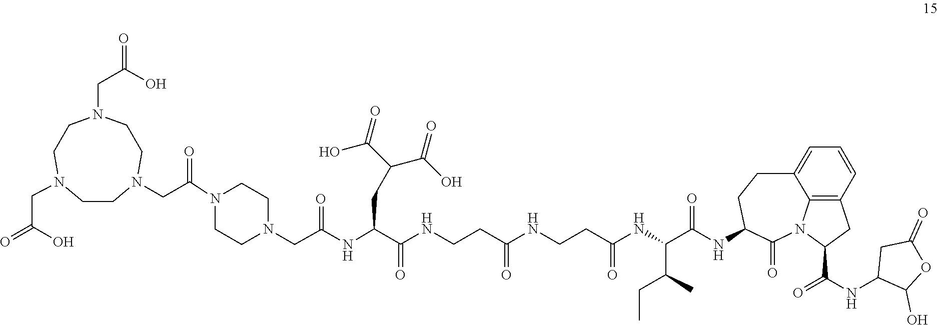 [SMILES: CC[C@H](C)[C@H](NC(=O)CCNC(=O)CCNC(=O)[C@H](CC(C(=O)O)C(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2-((2S)-2-(2-(4-(2-(4,7-bis(carboxymethyl)-1,4,7-triazonan-1-yl) acetyl)piperazin-1-yl) acetamido)-3-((3-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-3-oxopropyl)amino)-3-oxopropyl) malonic acid (20 mg) was obtained.

ES/MS m/z 1185.6 (M+H)⁺.

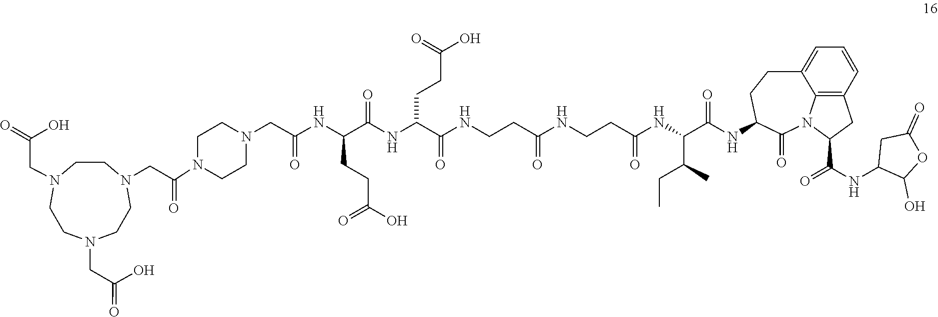 [SMILES: CC[C@H](C)[C@H](NC(=O)CCNC(=O)CCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,7R,18S,19S)-4,7-bis(2-carboxyethyl)-18-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-19-methyl-2,5,8,12,16-pentaoxo-3,6,9,13,17-pentaazahenicosyl)piperazin-1-yl)-2-oxoethyl)-1,4,7-triazonane-1,4-diyl)diacetic acid (30 mg) was obtained.

ES/MS m/z 1270.6 (M+H)⁺.

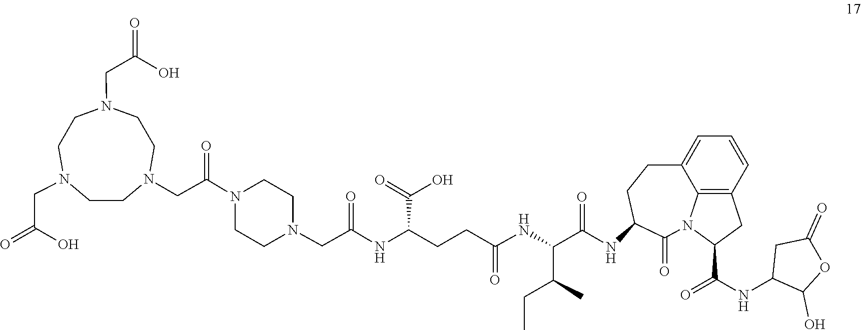 [SMILES: CC[C@H](C)[C@H](NC(=O)CC[C@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-(2-(4-(2-(((1S)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (30 mg) was obtained.

ES/MS m/z 999.5 (M+H)⁺.

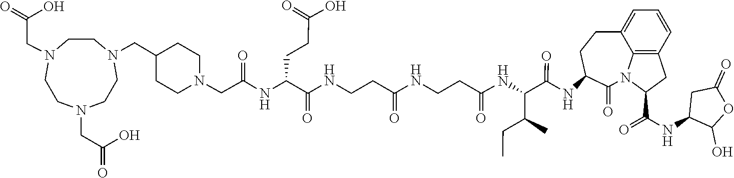 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)OC1O)C3]

### Example 2: Synthesis of Exemplary Compounds of Formula (II)

### General Procedure for AlF Complexation

To a reaction vial containing peptide precursor and a stir bar, was added equal equivalent (1.5-3.0 equiv relative to peptide) of 20 mM AlCl₃ in 0.1M NaOAc (PH˜4.5) and 100 mM NaF in H₂O. Then acetonitrile (0-34% of the total reaction volume) was added. The mixture was heated to 100° C. for 15-30 mins. Acetonitrile was removed under reduced pressure, and the aqueous solution was purified by either gold aq-C18 ISCO column or Phenomenex Gemini C18 RP-HPLC preparatory column (0.1% formic acid in water and acetonitrile as eluents). The proper fractions were collected and lyophilized to afford the peptide AlF complexes as white, fluffy solids.

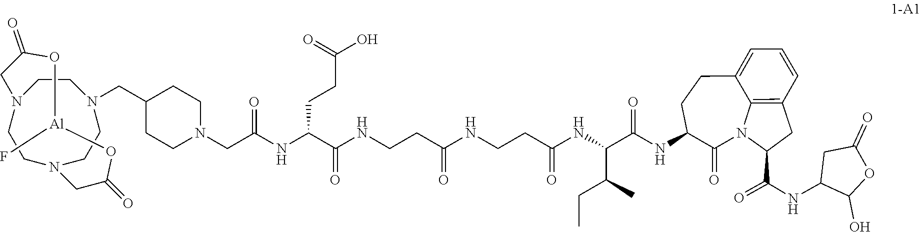

Following the general procedure for AlF complexation, 2,2′-(7-((1-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (15 mg) was converted to 2,2′-(7-((1-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) (10 mg) as a white, fluffy solid.

ES/MS m/z 1156.5 (M+H)⁺.

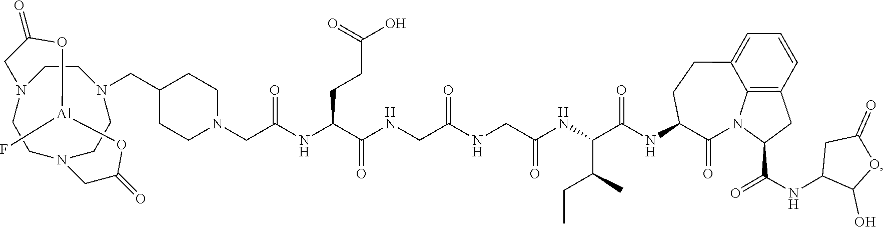

Following the general procedure for AlF complexation, 2,2′-(7-((1-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperidin-4-yl)methyl)-1,4,7-triazonane-1,4-diyl)diacetic acid (30 mg) was converted to 2,2′-(7-((1-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperidin-4-yl)methyl)-1,4,7-triazonane-1,4-diyl)diacetic acid aluminum fluoride complex (1:1) (21 mg) as a white, fluffy solid.

ES/MS m/z 1128.6 (M+H)⁺.

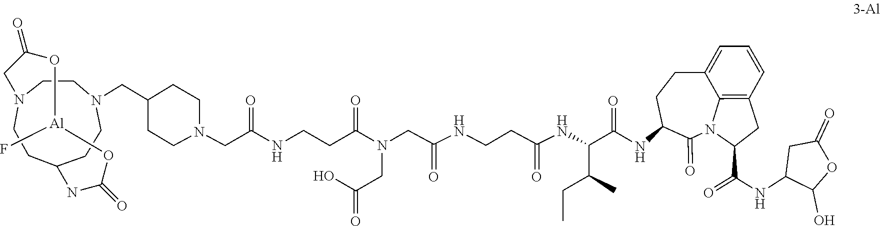

Following the general procedure for AlF complexation, 2,2′-(7-((1-((15S,16S)-7-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (15 mg) was converted to 2,2′-(7-((1-((15S,16S)-7-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid, aluminum fluoride complex (1:1) (12.5 mg) as a white, fluffy solid.

ES/MS m/z 1142.5 (M+H)⁺.

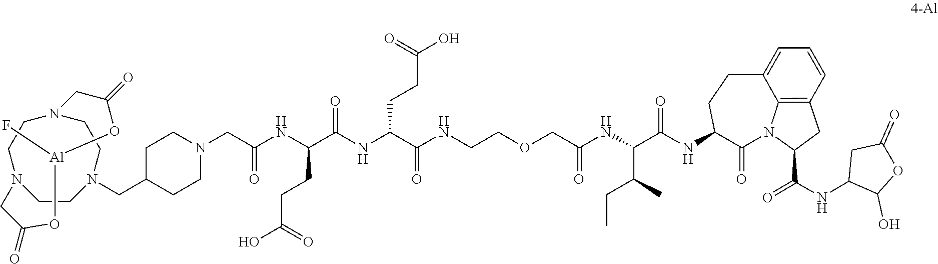

Following the general procedure for AlF complexation, 2,2′-(7-((1-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (20 mg) was converted to 2,2′-(7-((1-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid, aluminum fluoride complex (1:1) (18 mg) as a white, fluffy solid.

ES/MS m/z 1244.7 (M+H)⁺.

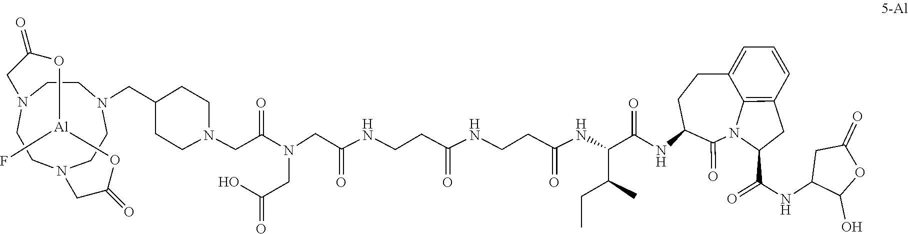

Following the general procedure for AlF complexation, 2,2′-(7-((1-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (10 mg) was converted to 2,2′-(7-((1-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) (9.9 mg) as a white, fluffy solid.

ES/MS m/z 1142.6 (M+H)⁺.

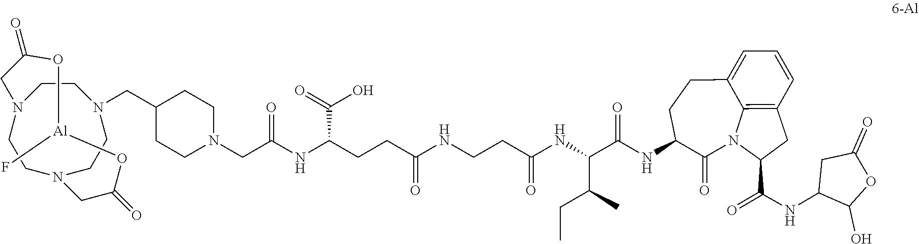

Following the general procedure for AlF complexation, 2,2′-(7-((1-(2-(((1S)-1-carboxy-4-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid (18 mg) was converted to 2,2′-(7-((1-(2-(((1S)-1-carboxy-4-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid, aluminum fluoride complex (1:1) (15 mg) as a white, fluffy solid.

ES/MS m/z 1085.5 (M+H)⁺.

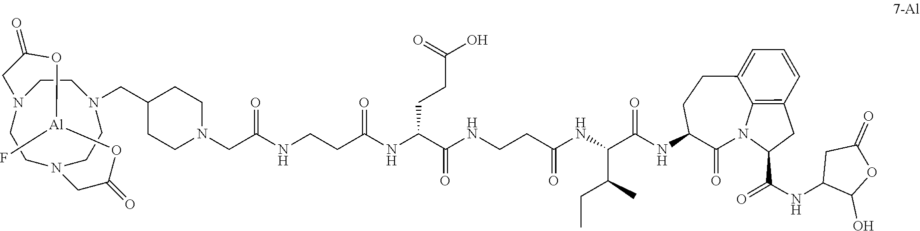

Following the general procedure for AlF complexation, 2,2′-(7-((1-((8R,15S,16S)-8-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (12 mg) was converted to 2,2′-(7-((1-((8R,15S,16S)-8-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid, aluminum fluoride complex (1:1) (9.6 mg) as a white, fluffy solid.

ES/MS m/z 1156.6 (M+H)⁺.

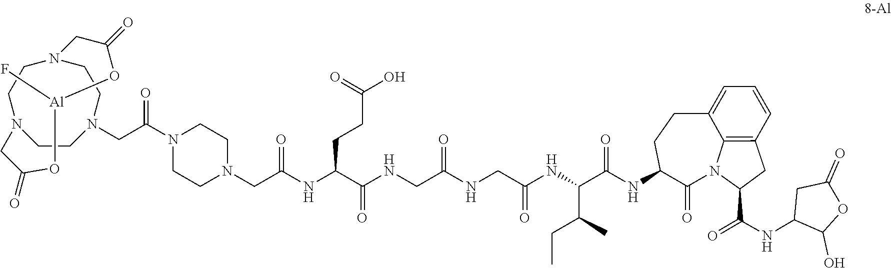

Following the general procedure for AlF complexation, 2,2′-(7-(2-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperazin-1-yl)-2-oxoethyl)-1,4,7-triazonane-1,4-diyl)diacetic acid (30 mg) was converted to 2,2′-(7-(2-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperazin-1-yl)-2-oxoethyl)-1,4,7-triazonane-1,4-diyl)diacetic acid, aluminum fluoride complex (1:1) (22 mg) as a white, fluffy solid.

ES/MS m/z 1157.5 (M+H)⁺.

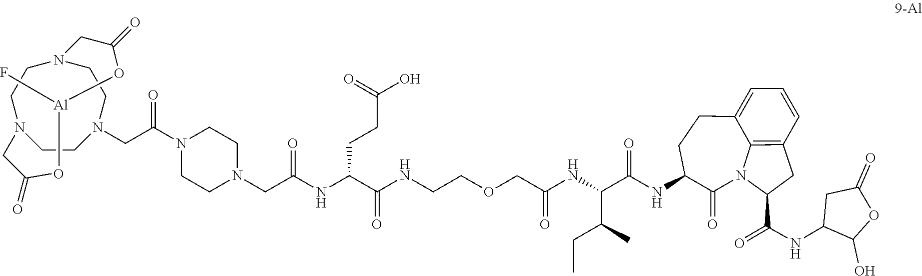

Following the general procedure for AlF complexation, 2,2′-(7-(2-(4-((4R,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,11-trioxo-9-oxa-3,6,12-triazahexadecyl)piperazin-1-yl)-2-oxoethyl)-1,4,7-triazonane-1,4-diyl)diacetic acid (48 mg) was converted to 2,2′-(7-(2-(4-((4R,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,11-trioxo-9-oxa-3,6,12-triazahexadecyl)piperazin-1-yl)-2-oxoethyl)-1,4,7-triazonane-1,4-diyl)diacetic acid, aluminum fluoride complex (1:1) (35 mg) as a white, fluffy solid.

ES/MS m/z 1144.5 (M+H)⁺.

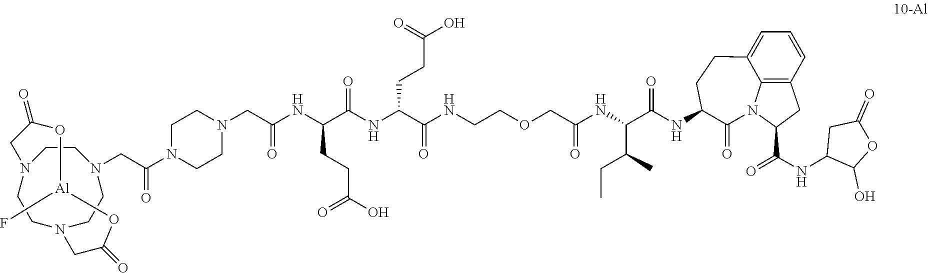

Following the general procedure for AlF complexation, 2,2′-(7-(2-(4-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperazin-1-yl)-2-oxoethyl)-1,4,7-triazonane-1,4-diyl)diacetic acid (15 mg) was converted to 2,2′-(7-(2-(4-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperazin-1-yl)-2-oxoethyl)-1,4,7-triazonane-1,4-diyl)diacetic acid, aluminum fluoride complex (1:1) (6.6 mg) as a white, fluffy solid.

ES/MS m/z 1273.5 (M+H)⁺.

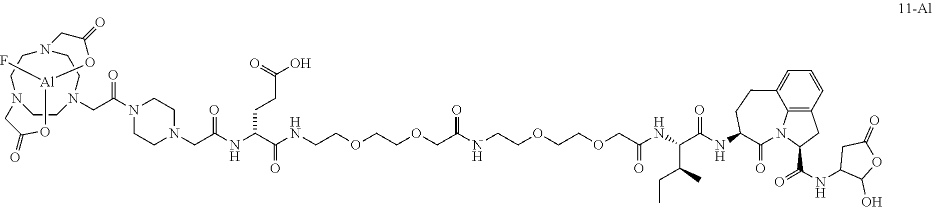

Following the general procedure for AlF complexation, 2,2′-(7-(2-(4-((4R,25S,26S)-4-(2-carboxyethyl)-25-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-26-methyl-2,5,14,23-tetraoxo-9,12,18,21-tetraoxa-3,6,15,24-tetraazaoctacosyl)piperazin-1-yl)-2-oxoethyl)-1,4,7-triazonane-1,4-diyl)diacetic acid (16 mg) was converted to 2,2′-(7-(2-(4-((4R,25S,26S)-4-(2-carboxyethyl)-25-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-26-methyl-2,5,14,23-tetraoxo-9,12,18,21-tetraoxa-3,6,15,24-tetraazaoctacosyl)piperazin-1-yl)-2-oxoethyl)-1,4,7-triazonane-1,4-diyl)diacetic acid, aluminum fluoride complex (1:1) (11 mg) as a white, fluffy solid.

ES/MS m/z 1333.6 (M+H)⁺.

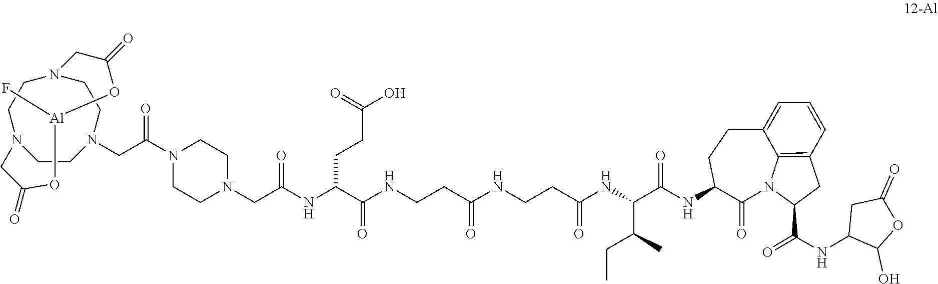

Following the general procedure for AlF complexation, 2,2′-(7-(2-(4-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (15 mg) was converted to 2,2′-(7-(2-(4-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid, aluminum fluoride complex (1:1) (10 mg) as a white, fluffy solid.

ES/MS m/z 1185.6 (M+H)⁺.

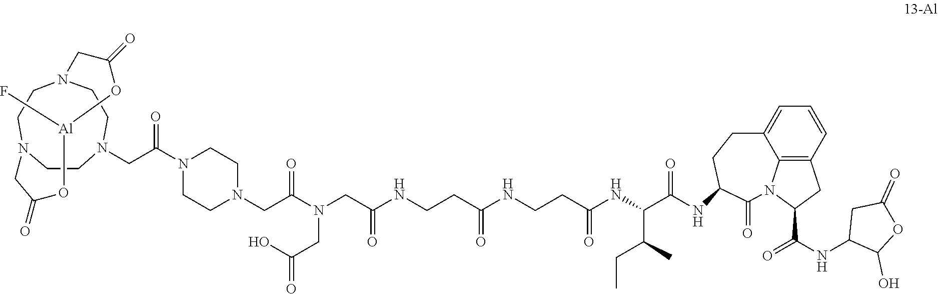

Following the general procedure for AlF complexation, 2,2′-(7-(2-(4-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (15 mg) was converted to 2,2′-(7-(2-(4-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid, aluminum fluoride complex (1:1) (6.6 mg) as a white, fluffy solid.

ES/MS m/z 1171.6 (M+H)⁺.

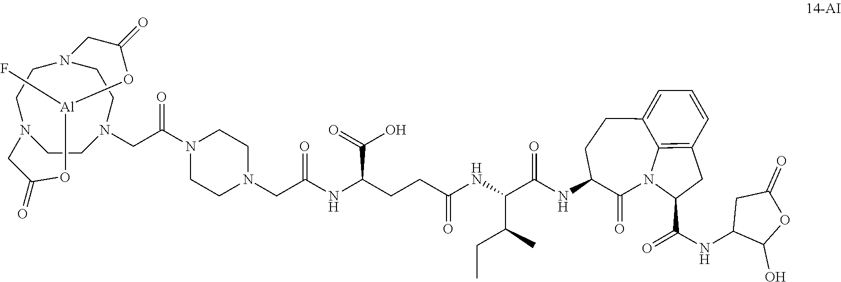

Following the general procedure for AlF complexation, 2,2′-(7-(2-(4-(2-(((1R)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (16 mg) was converted to 2,2′-(7-(2-(4-(2-(((1R)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid, aluminum fluoride complex (1:1) (11 mg) as a white, fluffy solid.

ES/MS m/z 1043.5 (M+H)⁺.

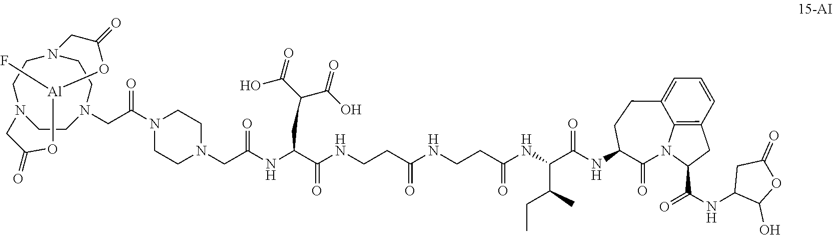

Following the general procedure for AlF complexation, 2-((2S)-2-(2-(4-(2-(4,7-bis(carboxymethyl)-1,4,7-triazonan-1-yl) acetyl)piperazin-1-yl) acetamido)-3-((3-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-3-oxopropyl)amino)-3-oxopropyl) malonic acid (15 mg) was converted to 2-((2S)-2-(2-(4-(2-(4,7-bis(carboxymethyl)-1,4,7-triazonan-1-yl) acetyl)piperazin-1-yl) acetamido)-3-((3-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-3-oxopropyl)amino)-3-oxopropyl) malonic acid, aluminum fluoride complex (1:1) (7.8 mg) as a white, fluffy solid.

ES/MS m/z 1229.6 (M+H)⁺.

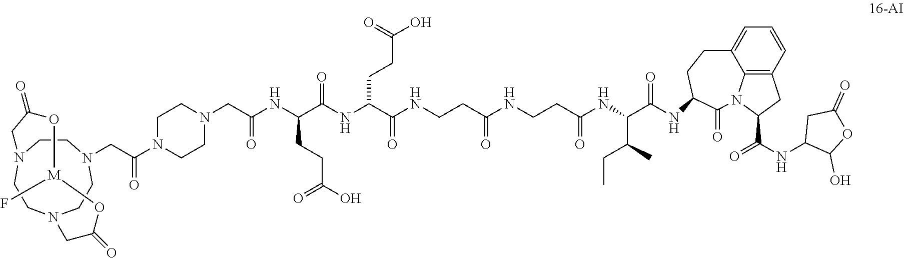

Following the general procedure for AlF complexation, 2,2′-(7-(2-(4-((4R,7R,18S,19S)-4,7-bis(2-carboxyethyl)-18-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-19-methyl-2,5,8,12,16-pentaoxo-3,6,9,13,17-pentaazahenicosyl)piperazin-1-yl)-2-oxoethyl)-1,4,7-triazonane-1,4-diyl)diacetic acid (17 mg) was converted 2,2′-(7-(2-(4-((4R,7R,18S,19S)-4,7-bis(2-carboxyethyl)-18-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-19-methyl-2,5,8,12,16-pentaoxo-3,6,9,13,17-pentaazahenicosyl)piperazin-1-yl)-2-oxoethyl)-1,4,7-triazonane-1,4-diyl)diacetic acid, aluminum fluoride complex (1:1) (13 mg) as a white, fluffy solid.

ES/MS m/z 1314.6 (M+H)⁺

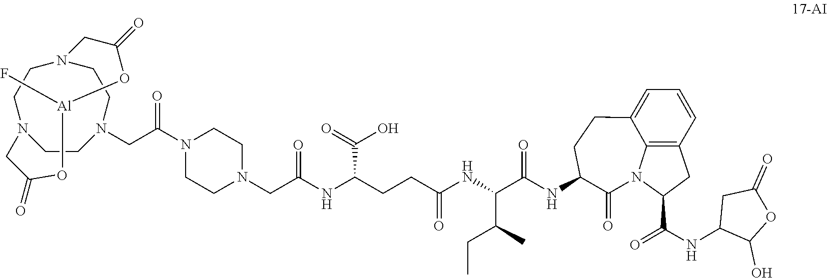

Following the general procedure for AlF complexation, 2,2′-(7-(2-(4-(2-(((1S)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (14 mg) was converted to 2,2′-(7-(2-(4-(2-(((1S)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid, aluminum fluoride complex (1:1) (9.7 mg) as a white, fluffy solid.

ES/MS m/z 1043.5 (M+H)⁺.

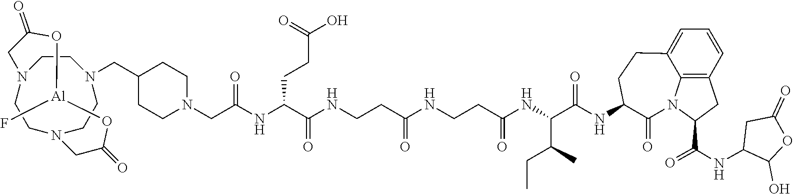

### Example 3: Radiosynthesis of ¹⁸F-Granzyme B (¹⁸F-GZB)

Typical ¹⁸F-GZB compound RCY ranges from 1.0-19.6% using 0.4-1.7 Ci starting activity with synthesis time of 75±10 minutes. Reaction vessel was preloaded with the reaction mixture containing precursor (e.g. 0.2-0.6 mg), AlCl₃·6H₂O (e.g. 34-82 μg), acetic acid/sodium acetate aqueous buffer (e.g. 0.2-0.4 mL, 1 M, pH 3-5), water (e.g. 0.1-0.3 mL)] and acetonitrile (e.g. 25-50% of total reaction mixture volume). The [¹⁸F]Fluoride activity was retained on a conditioned anion exchange resin (e.g. Sep-Pak Accell Plus QMA Carbonate Plus Light Cartridge, 46 mg sorbent per cartridge, 40 μm particle size, Waters part No. 186004540) and was then eluted into the reaction vessel using 0.9% saline (e.g. 0.5-0.8 mL). The resulting mixture was heated (e.g. 105° C.) for certain time (e.g. 15 minutes) and then cooled (e.g. 60° C.) prior to dilution with water (e.g. 4-5 mL). The resulting crude was loaded onto a semi-preparative reverse phase HPLC column (e.g. Agilent ZORBAX Eclipse XDB-C18, 5 μm, 9.4×250 mm, Part No. 990967-202) for purification [e.g. mobile phase comprising aqueous acetonitrile (8-15%) solution, pH 1-8]. The HPLC fraction containing the purified ¹⁸F-GZB compound was diluted with 0.5% w/v sodium ascorbate aqueous solution (e.g. 30-50 mL) and was then passed through a conditioned reverse phase cartridge (e.g. Sep-Pak® Light C18 Cartridge, 130 mg sorbent per cartridge, 55-105 μm particle size, Waters part No. WAT0523501). The retained ¹⁸F-GZB was washed with 0.5% w/v sodium ascorbate aqueous solution (e.g. 5-15 mL) and eluted off the cartridge using ethanol (e.g. 1-1.5 mL) into the formulation vial containing 0.5% w/v sodium ascorbate in 0.9% saline (e.g. 6-10 mL). The C18 cartridge was then rinsed with additional 0.5% w/v sodium ascorbate in 0.9% saline (e.g. 3-3.5 mL) and the rinsate was collected into the formulation vial. Certain amount of diluent (10% v/v ethanol and 90% v/v 0.9% saline containing 0.5% w/v sodium ascorbate) can be added to adjust product strength.

To prepare sterile product, the resulting product (¹⁸F-GZB in 10% v/v ethanol and 90% v/v 0.9% saline containing 0.5% w/v sodium ascorbate) was sterile filtered through a 0.22 μm filter (e.g. Millex® GV sterilizing filter, Millipore Part #SLGV033RS; Millex GV 25 mm sterilizing filter, Millipore Part #SLGVV255F; and Millex LG 25 mm sterilizing Filter, Millipore Part #SLLG025SS) into a bulk product vial.

### Example 4: Radiosynthesis of Compound ¹⁸F-1-Al

Typical Compound ¹⁸F-1-Al RCY synthesized on a ORA Neptis Perform radiosynthesizer ranges from 14-20% (n=7) using 29-55 GBq starting activity. Synthesis time is in 68±5 minutes with product radiochemical purity >93% and specific activity ranging from 167-1320 GBq/μmol (3.9-30.9 mCi/μg).

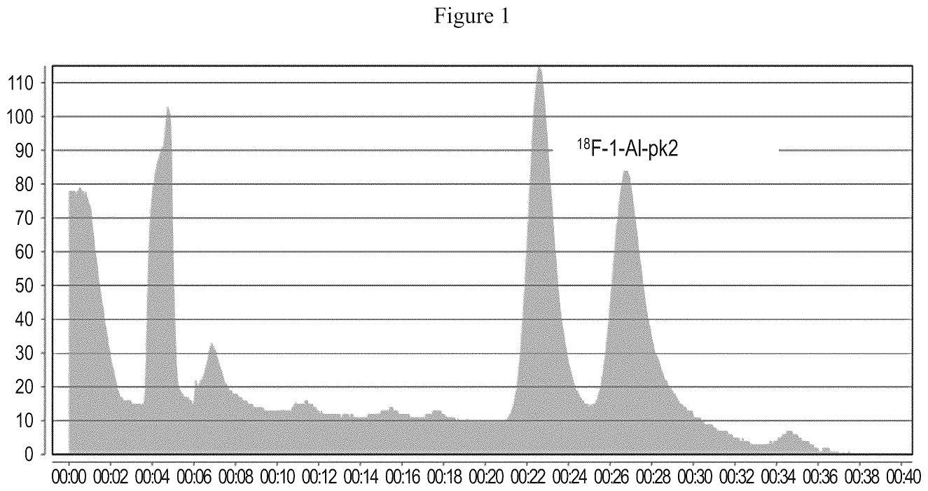

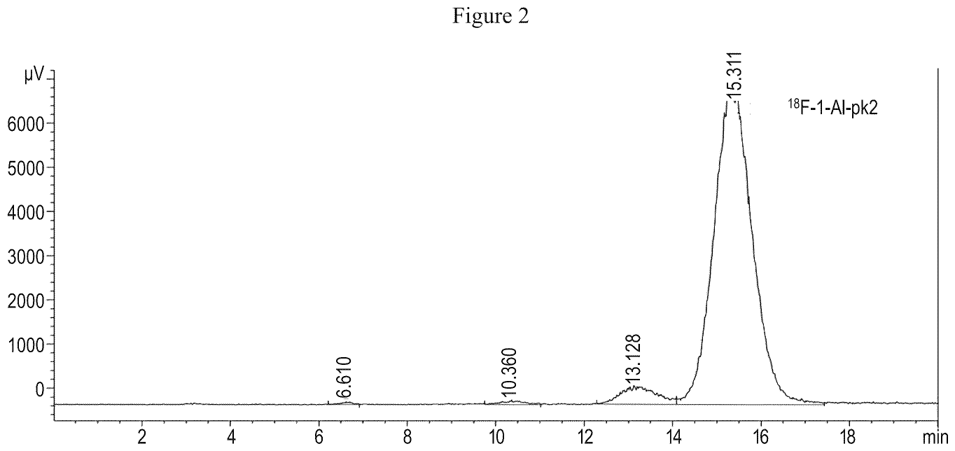

Reaction vessel was preloaded with precursor (0.22 mg), AlCl₃·6H₂O (38.6 μg), acetic acid/sodium acetate buffer (0.22 mL, 1M, pH=3.5), water (0.08 mL) and acetonitrile (0.8 mL). [¹⁸F]Fluoride was retained on a conditioned Waters Sep-Pak Accell Plus QMA Carbonate Plus Light Cartridge (46 mg sorbent per cartridge, 40 μm particle size, Waters part No. 186004540) and was then eluted into the reaction vessel using 0.9% saline (0.8 mL). The resulting mixture was kept at 105° C. for 15 minutes and then cooled down to 60° C. 4.6 mL of water was added to reaction vessel. The diluted reaction mixture was then loaded onto semi-preparative HPLC column (Agilent ZORBAX Eclipse XDB-C18, 5 μm, 9.4×250 mm, Part No. 990967-202) and purified using mobile phase comprising 89% 20 mM ammonium acetate aqueous solution (pH 7) and 11% acetonitrile with flowrate of 4 mL/min. Typical peak collection ranged from 26˜28 minutes. (FIG. 1) The collected fraction was then diluted with 0.5% (w/v) sodium ascorbate aqueous solution (˜30 mL) and passed through a conditioned Waters Sep-Pak C18 Plus Light cartridge (130 mg sorbent per cartridge, 55-105 μm, Part No: 023501). Product retained on the cartridge was then washed with sodium ascorbate aqueous solution (0.5%, w/v, ˜15 mL) and then eluted with 1 mL ethanol into final product vial containing 6 mL of 0.9% saline and sodium ascorbate (0.5% w/v). The C18 cartridge was then rinsed with additional 3 mL of 0.9% saline containing sodium ascorbate (0.5% w/v) to afford 10 mL formulated product as 10% v/v ethanol and 90% v/v 0.9% saline containing 0.5% w/v sodium ascorbate. A sample from the product vial was taken out for HPLC analysis. (FIG. 2)

### Analytical HPLC Condition:

Analytical Column: Agilent ZORBAX Eclipse XDB-C18 4.6×150 mm, Part No. 993967-902; flow rate=0.5 mL/min, UV@254 nm

Isocratic method—Mobile Phase: 87% 20 mM ammonium acetate in water and 13% acetonitrile (HPLC grade)

### Example 5: Radiosynthesis of Compound ¹⁸F-12-Al

Typical Compound ¹⁸F-12-Al synthesized on a ORA Neptis Perform radiosynthesizer ranges from 7-10% using 27-41 GBq starting activity. Synthesis time is 60±5 minutes with product radiochemical purity >95% and specific activity ranging from 237-1083 GBq/μmol (5.4-24.7 mCi/μg).

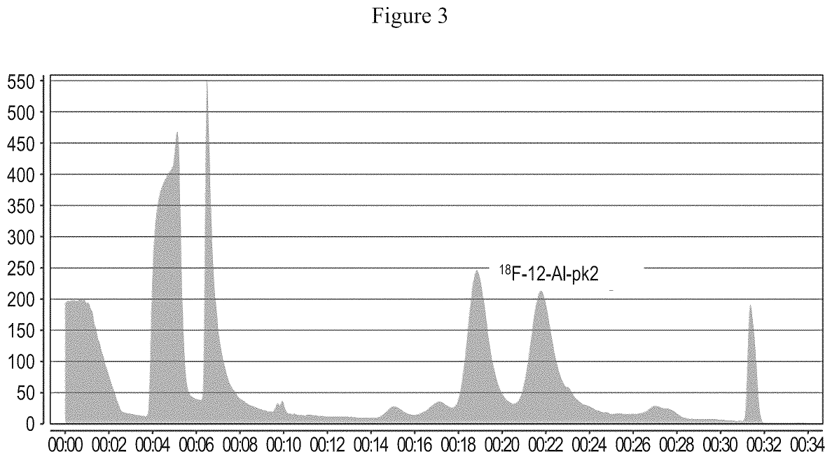

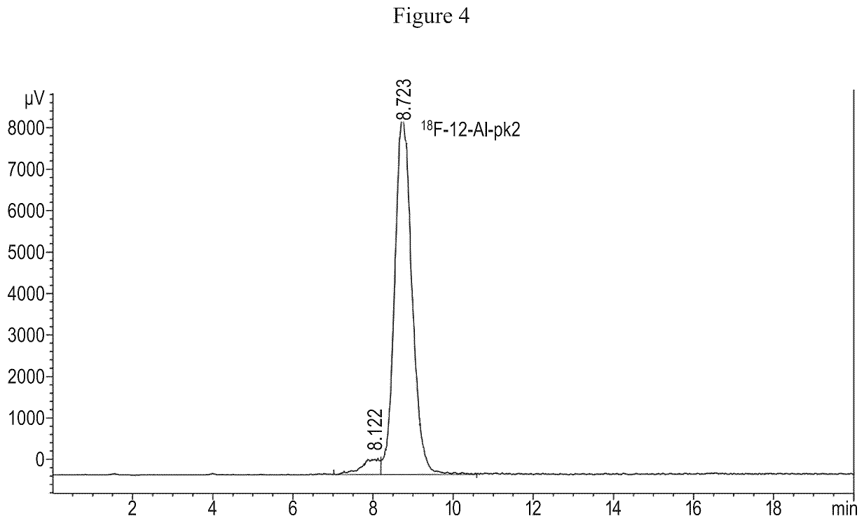

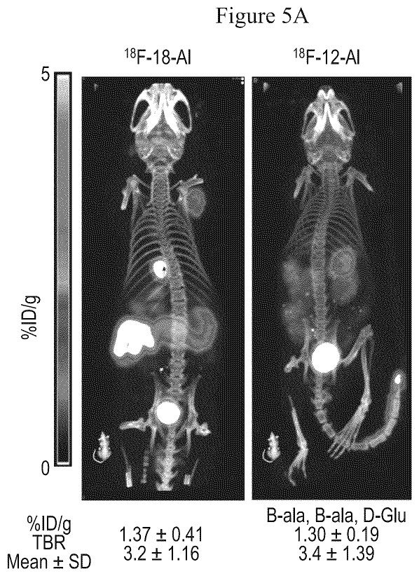

Reaction vessel was preloaded with precursor (0.2 mg), AlCl₃·6H₂O (38.6 μg), acetic acid/sodium acetate buffer (0.2 mL, 1M, pH=3.5), water (0.08 mL) and acetonitrile (0.8 mL). [¹⁸F]Fluoride was retained on a conditioned Waters Sep-Pak Accell Plus QMA Carbonate Plus Light Cartridge (46 mg sorbent per cartridge, 40 μm particle size, Waters part No. 186004540) and was then eluted into the reaction vessel using 0.9% saline (0.8 mL). The resulting mixture was kept at 105° C. for 15 minutes and then cooled down to 60° C. 4.6 mL of water was added to the reaction vessel. The diluted reaction mixture was then loaded onto semi-preparative HPLC column (Agilent ZORBAX Eclipse XDB-C18, 5 μm, 9.4×250 mm, Part No. 990967-202) and purified using mobile phase comprising 89% 20 mM ammonium acetate aqueous solution (pH 7) and 11% acetonitrile with flowrate of 4 mL/min. Typical peak collection ranged from 21˜23 minutes. (FIG. 3) The collected fraction was then diluted with 0.5% (w/v) sodium ascorbate aqueous solution (˜30 mL) and passed through a conditioned Waters Sep-Pak C18 Plus Light cartridge (130 mg sorbent per cartridge, 55-105 μm, Part No: 023501). Product retained on the cartridge was then washed with sodium ascorbate aqueous solution (0.5%, w/v, ˜15 mL) and then eluted with 1 mL ethanol into final product vial containing 6 mL of 0.9% saline and sodium ascorbate (0.5% w/v). The C18 cartridge was then rinsed with additional 3 mL of 0.9% saline containing sodium ascorbate (0.5% w/v) to afford 10 mL formulated product as 10% v/v ethanol and 90% v/v 0.9% saline containing 0.5% w/v sodium ascorbate. A sample from the product vial was taken out for HPLC analysis. (FIG. 4)

### Analytical HPLC Condition:

Analytical Column: Agilent ZORBAX Eclipse XDB-C18 4.6×150 mm, Part No. 993967-902; flow rate=1.0 mL/min, UV@254 nm

Gradient method—Mobile Phase: A: 20 mM Ammonium acetate in water, B: Acetonitrile (HPLC grade)

TABLE 7
Analytical HPLC Solvent Gradient

| Time (min) | % Mobile Phase A | % Mobile Phase B |
|---|---|---|
| 0.0 | 90 | 10 |
| 15.0 | 85 | 15 |

### Example 6: Competitive Binding Assay of Human Granzyme B

Purpose: To perform competitive binding assays with human granzyme B using Compound ¹⁸F-21-Al to determine IC₅₀ values of potential granzyme B ligands

Initial Plate Preparation (including total binding and non-specific binding):
1. 78 μL of reaction buffer, as described above, is added to each well in a 96 well plate
2. For wells used to examine total binding (defined as the binding of the radioligand in the absence of competitors), 2 μL of DMSO is added to give 1% final DMSO concentration for all wells
3. For wells used to examine non-specific binding (defined as the binding of the radioligand in the presence of 10 μM 21), 2 μL of 1 mM Compound 21 is added to give a final concentration of 10 μM

### Enzyme Preparation:

1. Granzyme B (GZB, human lymphocyte) is diluted to 0.5 μg/mL using reaction buffer as described above
2. 100 μL of 0.5 μg/mL diluted human GzB is added to each well, giving 50 ng per well and a final concentration of 0.25 μg/mL

### Compound Preparation:

1. DMSO or water is added to the respective compound vial to make 1 mM compound stock solution
2. 1 mM compound stock solution is serially diluted by ½ log (3.16 fold) by adding 18.5 μL of compound stock to 40 μL of DMSO, mixing by pipetting up and down, and transferring 18.5 μL of the mixture to 40 μL of DMSO. This process is repeated to create 10 dilution points for test compounds.
3. For wells used to examine ligand binding competition, 2 μL of each dilution is dispensed into the 96 well plates prepared as described above, giving a further dilution of 1/100.

### Radioligand Preparation:

1. 200 nM of Compound ¹⁸F-21-Al (10× radioligand stock) is prepared in reaction buffer, aiming for approximately 2 million CPM per 20 μL of solution (input).
2. 20 μL of radioligand stock is dispensed into each well to give a final concentration per well of 20 nM.

### Incubation Conditions, Post-Incubation Sample Processing and Data Analysis:

1. All 96 wells should have a final volume of 200 μL, and the assay plates are incubated at 37° C. for 90 minutes.
2. All samples from the assay plates are transferred to MultiScreen_{HTS} FB plates that have been presoaked in PBS buffer, pH 7.4, and filtered using a vacuum manifold. An additional 150 μL of PBS buffer is added to each well in the assay plates and combined to ensure transfer of any remaining samples
3. MultiScreen_{HTS} FB plates are washed 3× with 150 μL of PBS buffer.
4. All filters are separated and transferred to individual tubes using Multiscreen punch tips and the Millipore multiple punch apparatus
5. The sample set is analyzed using the Wizard 2480 automatic gamma-counter [Perkin Elmer] through a [¹⁸F]-specific profile. Values are reported as decay-corrected counts per minute (CPM).
6. Resulting CPM values are normalized and converted to % inhibition by the following equation:

$$\% inhibition = \frac{1 - ( sample CPM - NSB CPM )}{TB CPM - NSB CPM}$$

*the total bound fraction is typically less than 10% of the added radioligand under such assay conditions.
Resulting % inhibition values were plotted using the software GraphPad Prism 8.4.3 using the One site—Fit log IC₅₀ equation to determine the IC₅₀ value for each ligand.

$$Y = A + ( B - A ) / ( 1 + {10}^{( x - LogIC 50 )} )$$

Y=% Inhibition
X=Log concentration of the cold competing ligand (M)
A=minimum Y (0%)
B=maximum Y (100%)
Log IC₅₀=Log concentration of cold competing ligand (M) at half-way between minimum and maximum Y

### Results:

TABLE 8
IC₅₀ values of competitor ligands tested in GZB IC₅₀ binding assays
with Compound ¹⁸F-21-Al. For studies that were conducted more than
once, the average IC₅₀ values are reported with standard error.

| Competitor Ligand | IC₅₀ Value (nM) |
|---|---|
| Piperidine Series | |
| 1-Al | 5.7 |
| 2-Al | 7.5 |
| 3-Al | 4.7 |
| 4-Al | 2.8 |
| 5-Al | 5.7 |
| 6-Al | 4.5 |
| 7-Al | 6.7 |
| Piperazine Series | |
| 8-Al | 6.5 |
| 9-Al | 3.5 ± 0.78 (n = 2) |
| 10-Al | 3.6 |
| 11-Al | 2.4 |
| 12-Al | 4.2 |
| 13-Al | 4.2 |
| 14-Al | 2.7 |
| 15-Al | 2.5 |
| 16-Al | 4.2 |
| 17-Al | 2.7 |

### Example 7: In Vivo Imaging of ¹⁸F-Granzyme B Tracers in a Matrigel Mouse Model of Active and Pro-Form Granzyme B

This example explores the in vivo imaging activity of exemplary granzyme B-binding compounds disclosed herein.

Female nude athymic (5-6 weeks, 15-30 g) mice were purchased from Jackson Laboratories. Both granzyme B (Human lymphocytes, Enzo Life Sciences) and inactivated human pro-form granzyme B (R&D Systems) were also commercially purchased. On the day of the imaging study, each mouse had a cannula inserted into the lateral tail vein (SAI 27 g butterfly with 12 cm tubing, #BF27-01) to allow for intravenous radiotracer administration. Each granzyme B enzyme was then brought up to a final concentration of 0.05 μg/ul using phosphate buffered saline (GE, Hyclone). Matrigel (65 μl, Corning) was mixed with 15 μl of 0.75 μg of each granzyme B enzyme (granzyme B and pro-form granzyme B) within each Eppendorf tube. The mice were then anesthetized with 2.5-3% isoflurane mixed with oxygen. Approximately 60-80 μl of granzyme B/Matrigel or the pro-form granzyme B/Matrigel were then injected using a 28-gauge X 0.5-inch insulin syringe (Terumo) to form implants on the right and left shoulder flanks of the mice. Approximately five minutes later, the mice were administered the ¹⁸F-radiotracer via a bolus intravenous injection (approximately 150 μCi in a total volume of 100 ul saline plus an additional ˜25 ul saline to flush the catheter line). After the radiotracer injection, the catheter was removed and measured for any remaining residual radioactivity. The mice were then placed back in their cage for recovery. A nanoScan® PET/CT (Mediso, Hungary) was used for micro-PET/CT imaging in which 15-minute static PET scans were conducted 75-minutes post-injection of the radiotracer. Tera-Tomo™ 3D PET iterative reconstruction along with scatter correction were then conducted post-acquisition. A short high-resolution computed tomography (CT) scan was also performed immediately after to allow for anatomical registration. PET signal in the granzyme B implants were quantified by manually drawing regions of interest (ROIs) over the Matrigel implants based on the fused PET/CT images and the corresponding activity values were determined with VivoQuant (Invicro, Massachusetts). All values were represented as % injected dose per gram (% ID/g). Target to background ratios (TBR's) were then calculated by dividing the granzyme B % ID/g value by the pro-form granzyme B % ID/g value. PET imaging timepoints and granzyme B concentrations used in these experiments were determined in previous studies (data not shown).

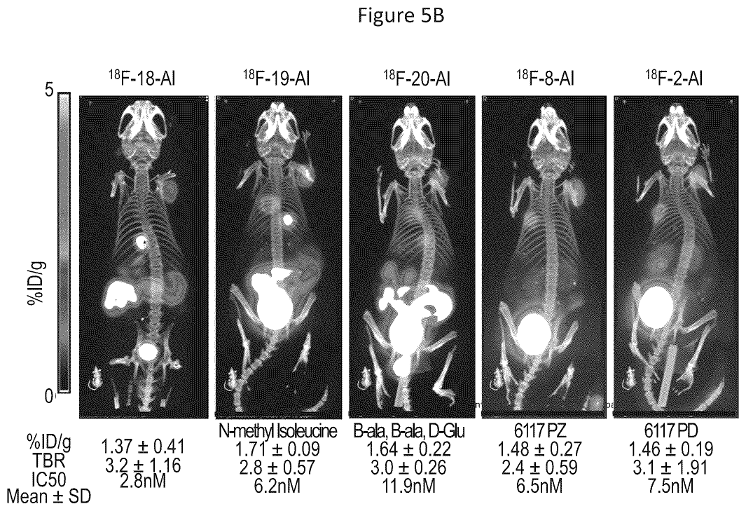

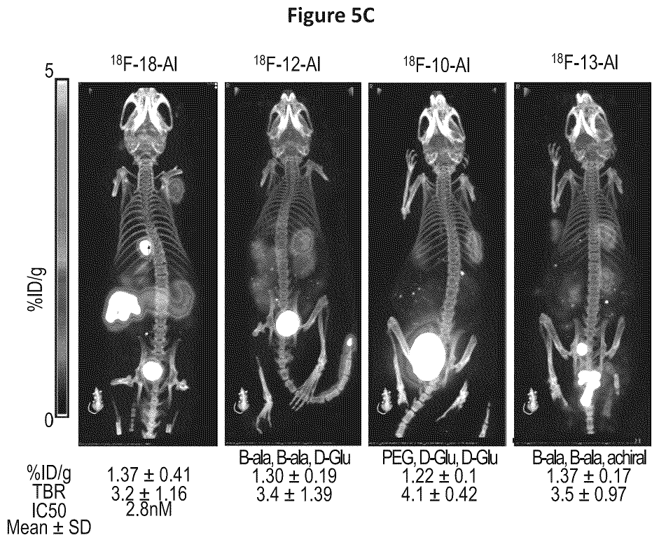

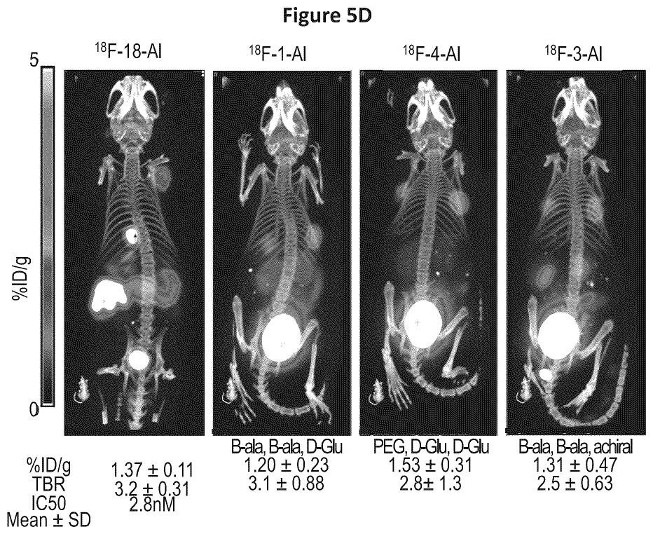

The imaging results are provided in FIGS. 5A-5D. The exemplary compounds examined in this example, including Compounds ¹⁸F-1-Al, ¹⁸F-2-Al, ¹⁸F-3-Al, ¹⁸F-4-Al, ¹⁸F-8-Al, ¹⁸F-10-Al, ¹⁸F-12-Al, and ¹⁸F-13-Al, showed good imaging activity in the Matrigel animal model. No significant gut uptake was observed in mice administering with exemplary compounds, while reference Compound ¹⁸F-18-Al showed a certain level of gut intake. Further, the exemplary compounds showed predominant renal clearance. In mice treated with the exemplary compounds (specifically ¹⁸F-1-Al, ¹⁸F-3-Al and ¹⁸F-4-Al, as shown in Table 8), no radiometabolites were observed in urine at 60 min post dose.

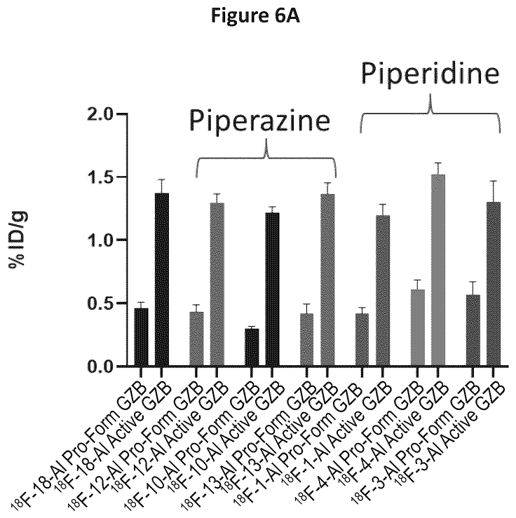

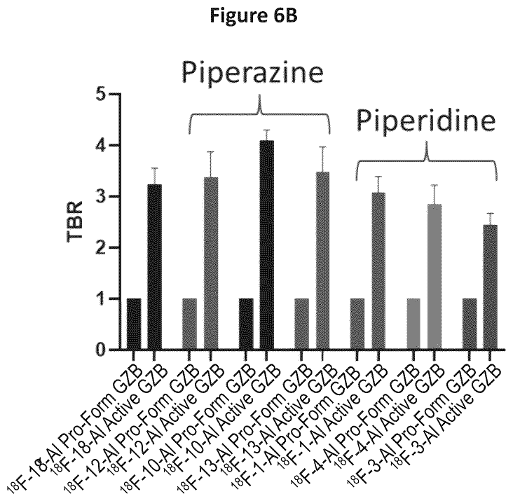

Further, FIG. 6 shows quantified values of % ID/g and TBR of representative compounds having either the piperazine linking ring or the piperidine linking ring.

### Example 8: Metabolism of Piperidine and Piperazine Compounds

Radioactive metabolite screening studies were performed by injection of [18F] granzyme B tracers in male CD-1 mice followed by analysis of urine samples at 60 minutes by radiometric HPLC.

Briefly, male CD-1 mice were anesthetized prior to dosing with approximately 250 μCi [¹⁸F] granzyme B tracer via tail vein injection (n=3 mice). Urine samples were collected at the 60 minutes post-injection following anesthesia and cervical dislocation. Samples were diluted 100-fold in water prior to injection by HPLC.

Diluted samples were then analyzed on an Agilent 1290 series UPLC UV coupled with a BGO coincidence detector. The HPLC method utilized either 1) Phenomenex Kinetex EVO C18 (150×2.1 mm, 1.7 micron); 100 μL injection volume; flow-rate: 0.5 mL/min; solvent for A: 0.1% formic acid in water; solvent for B: 100% acetonitrile; gradient: initial hold at 5% B hold for 1 min, 5% to 40% in 7 min, hold 40% B for 2 min, increase from 40% to 95% B in 1 min, hold 95% B for 2 min, and then return to 5% B to reequilibrate, or 2) Phenomenex Monolithic C18 (100×4.6 mm); 100 μL injection volume; flow-rate: 1.2 mL/min; solvent for A: 20 mM ammonium acetate in water; solvent for B: 100% methanol; gradient: same as above.

Table 9 provides the results from this example, reported as percent parent remaining.

TABLE 9
Percent parent remaining in 60-minute male CD-1 mouse urine.

| Compound | 60 minutes (% parent in urine) |
|---|---|
| ¹⁸F-1-Al | 100 |
| ¹⁸F-3-Al | 100 |
| ¹⁸F-4-Al | 100 |
| ¹⁸F-5-Al | 100 |
| ¹⁸F-6-Al | 66.3 |
| ¹⁸F-18-Al | 26.2 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A compound, or a pharmaceutically acceptable salt thereof, wherein the compound is of formula (I): 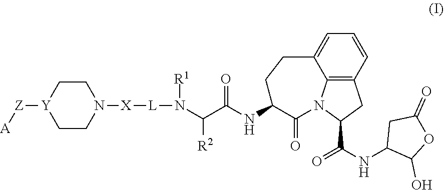
pharmaceutically acceptable wherein:
A is a chelating moiety;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Y is CH or N;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl; and
R² is C₁₋₆ alkyl or C₃₋₆ cycloalkyl.

2. The compound, or the pharmaceutically acceptable salt thereof, of claim 1, wherein the compound is of formula (Ia): 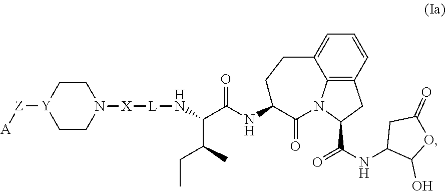
and wherein A, Z, Y, X, and L are each defined as in claim 1.

3. The compound, or the pharmaceutically acceptable salt thereof, of claim 1, wherein the compound is of formula (Ib): 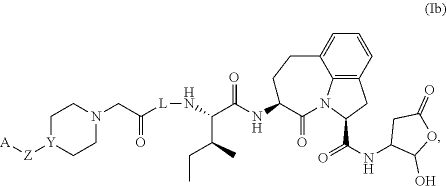
and wherein A, Y, Z, and L are each defined as in claim 1.

4. The compound, or the pharmaceutically acceptable salt thereof, of any one of claims 1-3, wherein L has 1-5 amino acid residues, inclusive; optionally wherein L includes one or more non-naturally occurring amino acid residues.

5. The compound, or the pharmaceutically acceptable salt thereof, of any one of claim 1-4, wherein L has an amino acid sequence selected from group consisting of:
Glu-Gly-Gly,
Glu-βAla-βAla,
γGlu,
_{D}γGlu,
γGlu-βAla,
_{D}Glu-βAla-βAla,
_{D}Glu-AEA,
_{D}Glu-AEEA-AEEA,
_{D}Glu-_{D}Glu-AEA,
_{D}Glu-_{D}Glu-βAla-βAla,
βAla-_{D}Glu-βAla,
Diacid-βAla-βAla,
N-acid-βAla-βAla, and
βAla-N-acid-βAla.

6. The compound, or a pharmaceutically acceptable salt thereof of any one of claims 1-5, wherein the chelating moiety A is 1,4,7-triazacyclononane-N,N′,N″-triacetic acid (NOTA) or 1,4,7-triazacyclononane-4,7-diyl diacetic acid (NODA).

7. The compound, or the pharmaceutically acceptable salt thereof any one of claims 1-6, wherein the compound is of formula (Ib-A): 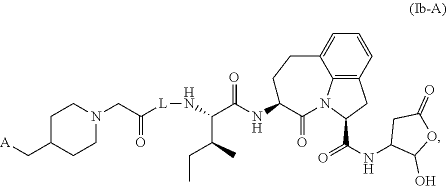
and wherein A and L each are defined in any one of claims 1-6.

8. The compound, or the pharmaceutically acceptable salt thereof, claim 7, wherein the compound is selected from the group consisting of Compounds 1-7.

9. The compound, or the pharmaceutically acceptable salt thereof, of claim 7, wherein the compound has the following structure:

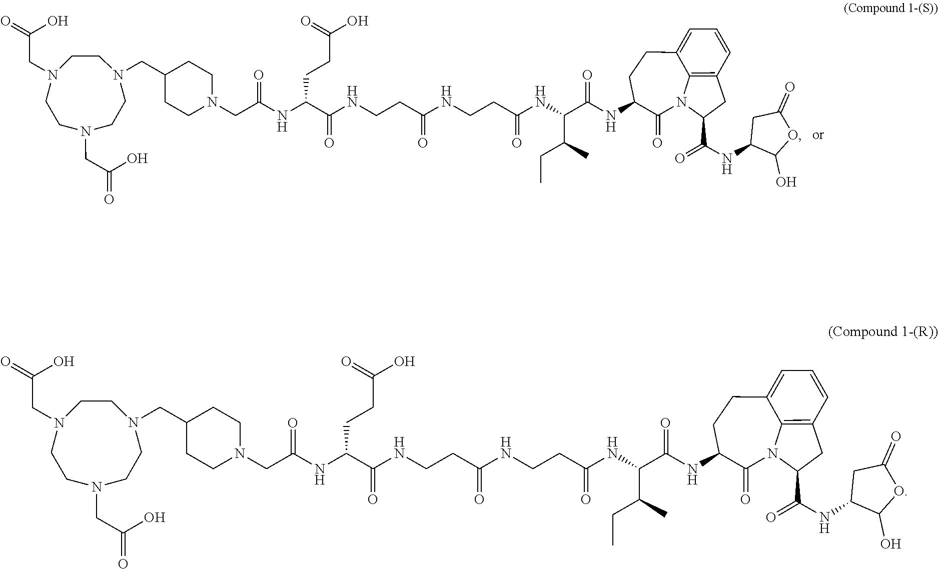 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)OC1O)C3 ; CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)OC1O)C3]

10. The compound, or the pharmaceutically acceptable salt thereof, of any one of claims 1-6, wherein the compound is of formula (Ib-B): 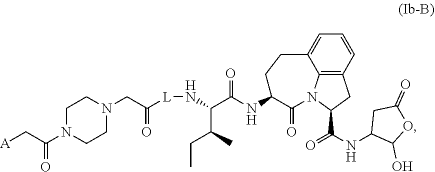
and wherein A and L each are defined in any one of claims 1-6.

11. The compound, or the pharmaceutically acceptable salt thereof, of claim 10, wherein the compound is selected from the group consisting of Compounds 8-17.

12. A compound, or a pharmaceutically acceptable salt thereof, wherein the compound is of formula (II): 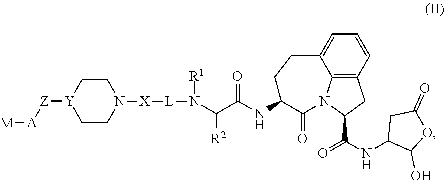
wherein:
M is a metal or a metal linked to a radioisotope;
A is a chelating moiety chelating the metal;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Y is CH or N;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl; and
R² is C₁₋₆ alkyl or C₃₋₆ cycloalkyl.

13. The compound, or the pharmaceutically acceptable salt thereof of claim 12, wherein the compound is of formula (IIa): 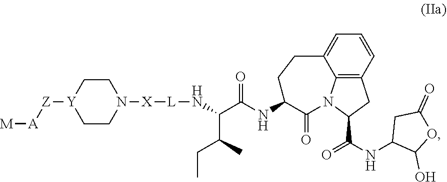
and wherein M, A, X, Y, Z, and L are each defined as in claim 12.

14. The compound, or the pharmaceutically acceptable salt thereof of claim 12, wherein the compound is of formula (IIb): 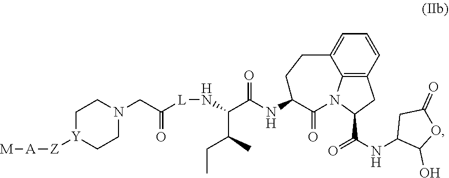
and wherein M, A, Y, Z, and L are each defined as in claim 13.

15. The compound, or the pharmaceutically acceptable salt thereof of any one of claims 12-14, wherein L has 1-5 amino acid residues, inclusive; optionally wherein L includes one or more non-naturally occurring amino acid residues.

16. The compound, or the pharmaceutically acceptable salt thereof of any one of claims 12-15, wherein L has an amino acid sequence selected from group consisting of:
Glu-Gly-Gly,
Glu-βAla-βAla,
γGlu,
_{D}γGlu,
γGlu-βAla,
_{D}Glu-βAla-βAla,
_{D}Glu-AEA,
_{D}Glu-AEEA-AEEA,
_{D}Glu-_{D}Glu-AEA,
_{D}Glu-_{D}Glu-βAla-βAla,
βAla-_{D}Glu-βAla,
Diacid-βAla-βAla,
N-acid-βAla-βAla, and
βAla-N-acid-βAla.

17. The compound, or the pharmaceutically acceptable salt thereof of any one of claims 12-16, wherein the chelating moiety A is 1,4,7-triazacyclononane-N,N′,N″-triacetic acid (NOTA) or 1,4,7-triazacyclononane-4,7-diyl diacetic acid (NODA).

18. The compound, or the pharmaceutically acceptable salt thereof of any one of claims 12-17, wherein M is non-toxic.

19. The compound, or the pharmaceutically acceptable salt thereof of any one of claims 12-18, wherein the metal is a radioisotope of Gallium (Ga), which optionally is ⁶⁸Ga.

20. The compound of claim 19, wherein the radioisotope of Gallium (Ga) is ⁶⁸Ga.

21. The compound, or the pharmaceutically acceptable salt thereof of any one of claims 12-18, wherein the metal is Aluminum (Al), which is linked to the radioisotope, which optionally is ¹⁸F.

22. The compound of claim 21, wherein the Aluminum (Al) is linked to ¹⁸F.

23. The compound, or a pharmaceutically acceptable salt thereof of claim 12, wherein the compound is selected from the group consisting of Compounds 1-Al to 17-Al.

24. The compound, or the pharmaceutically acceptable salt thereof, of claim 12, wherein the compound has the following structure:

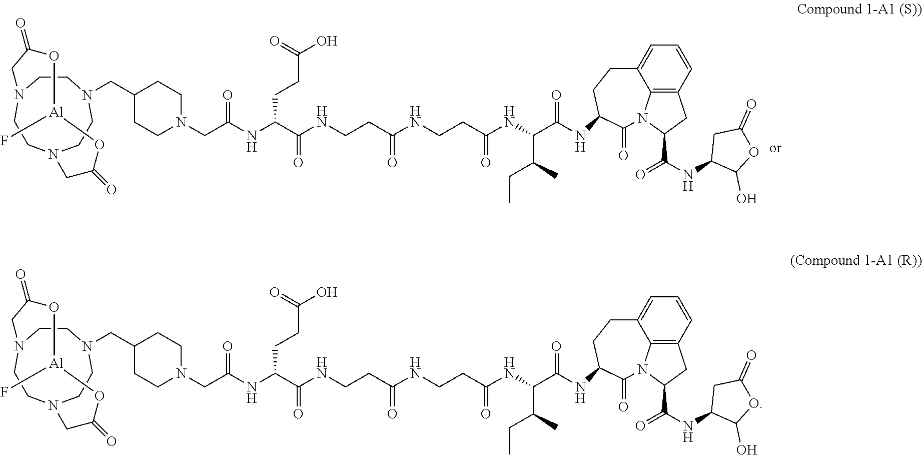

25. A pharmaceutical composition comprising a compound of any one of claims 12-24, a stereoisomer thereof, or a pharmaceutically acceptable salt thereof, and a pharmaceutically acceptable carrier.

26. A method of imaging granzyme B in a tissue, the method comprising:
(i) contacting the compound or the pharmaceutically acceptable salt thereof, of any one of claims 12-24, with a tissue suspected of comprising granzyme B, and
(ii) imaging the tissue based on radioisotope signals released from the compound or the pharmaceutically acceptable salt thereof.

27. The method of claim 26, optionally wherein the radioisotope contained in the compound or the pharmaceutically acceptable salt thereof, is ¹⁸F or ⁶⁸Ga.

28. The method of claim 26, wherein the contacting step is performed by administering the compound, or the pharmaceutically acceptable salt thereof, to a subject in need thereof.

29. The method of claim 28, wherein the subject is on a treatment for an immunoregulatory abnormality.

30. The method of claim 29, wherein the immunoregulatory abnormality is selected from the group consisting of an autoimmune disorder, an inflammatory disorder, a skin disorder, a cancer, and a cardiovascular disorder.

31. The method of claim 30, optionally wherein the immunoregulatory abnormality is a cancer.

32. The method of claim 29, wherein treatment comprises one or more additional therapeutic agents, which are selected from the group consisting of anti-inflammatory agents, steroids, immunotherapy agents, chemotherapeutic agents, and therapeutic antibodies.

33. The method of any one of claims 26-32, further comprising monitoring an immune response in the subject based on the imaging of granzyme B.
